(19) United States
(12) Patent Application Publication
(10) Pub. No.: US 2024/0043300 A1
(43) Pub. Date: Feb. 8, 2024

(54) SYSTEM AND METHOD FOR HYDRODYNAMIC CULTIVATION OF SEED OXYGENIC PHOTOGRANULES

(71) Applicant: University of Massachusetts, Boston, MA (US)

(72) Inventors: Joseph Gikonyo, Belchertown, MA (US); Chul Park, Amherst, MA (US)

(21) Appl. No.: 18/255,661

(22) PCT Filed: Dec. 3, 2021
(86) PCT No.: PCT/US2021/072725

§ 371 Date: Jun. 2, 2023

Related U.S. Application Data
(60) Provisional application No. 63/121,624, filed on Dec. 4, 2020.

(51) Int. Cl. C02F 3/32; C12N 1/12; C12M 1/00; C12M 1/06

(52) U.S. Cl. CPC C02F 3/325; C12N 1/12; C12M 21/02; C12M 27/02

(57) ABSTRACT

A method comprises placing in a vessel a mixture having a specified suspended solids or sludge concentration and comprising a water-based reaction medium and at least one microalgae including filamentous cyanobacteria, said water-based reaction medium comprising a nutrient material that is consumable by a live bacterium or by a live protozoan present in said water-based reaction medium, and incubating said mixture for a specified incubation period under at least intermittent illumination with a specified luminous flux during periods of illumination while mixing said mixture under a specified shear stress, wherein said filamentous cyanobacteria forms a supporting matrix that incorporates said live bacterium or said live protozoan into a biologically-active bioaggregate granule, wherein said incubating produces a plurality of said biologically-active bioaggregate granules.

### CROSS-REFERENCE TO RELATED APPLICATIONS

This application claims the benefit of priority to U.S. Provisional Patent Application Ser. No. 63/121,624 entitled “SYSTEM AND METHOD FOR HYDRODYNAMIC CULTIVATION OF SEED OXYGENIC PHOTOGRANULES,” filed Dec. 4, 2020, the disclosure of which is incorporated by reference herein in its entirety.

### BACKGROUND

Wastewater treatment is an energy-intensive procedure, in particular for aerobic wastewater treatment to remove sewage organic matter, which requires a substantial energy input to aerate the wastewater in order to dissolve oxygen gas (O₂). It is typical in the United States for wastewater treatment takes up as much as 25% of the total energy usage for many municipalities. Of this energy usage, about 60% is dedicated to wastewater aeration to support aerobic oxidation of organic matter and nitrogen. The aerobic process often involves the growth of floc-based biomass that requires large sedimentation basins to separate the biomass from water. In short, current wastewater treatment systems require substantial operating costs and capital investment.

Algae-based wastewater treatment has been gaining acceptance as an alternative to conventional wastewater treatment practices because it has the potential to treat wastewater without aeration through the symbiotic growth of microbes and oxygenic photosynthetic microalgae while still preserving the chemical energy within the wastewater in the form of grown biomass. A successful microalgae process could substantially reduce energy usage for wastewater treatment and could enhance recovery of chemical energy from the wastewater as a biofeedstock.

However, engineering challenges limit the adoption of microalgae processes. For example, photosynthetic microalgae do not typically naturally aggregate, which can make it difficult to separate the microalgae from the wastewater reaction medium, which can make biomass recycling and harvesting difficult. In addition, it can be challenging to configure the reactor system to produce sufficient amounts of the symbiotic microbes and oxygenic phototrophs for large-scale wastewater treatment within a reasonably short period of time. For example, the microalgae's need for light for photosynthesis has made only certain reactor configurations, such as large open ponds, useful for microalgae processes. This has limited the ability to adopt microalgae-based processing of wastewater to rural, suburban, and small community-based municipalities.

### SUMMARY

According to one aspect of the present disclosure, a method of cultivating oxygenic photogranules is described. In an example, the method includes the steps of placing in a vessel a mixture having a specified suspended solids or sludge concentration and comprising a water-based reaction medium and at least one microalgae including filamentous cyanobacteria, the water-based reaction medium comprising a nutrient material that is consumable by a live bacterium or by a live protozoan present in the water-based reaction medium, and incubating the mixture that has the specified suspended solids or sludge concentration for a specified incubation period under at least intermittent illumination with a specified luminous flux during periods of illumination while mixing the mixture under a specified shear stress, wherein the filamentous cyanobacteria forms a supporting matrix that incorporates the live bacterium or the live protozoan into a biologically-active bioaggregate granule, wherein the incubating produces a plurality of the biologically-active bioaggregate granules. The method can also include recovering at least a portion of the plurality of biologically-active bioaggregate granules from the incubated mixture.

According to another aspect of the present disclosure, a system for cultivating oxygenic photogranules for wastewater treatment is described. In an example, the system includes a reaction vessel for receiving a mixture comprising a water-based reaction medium having a specified suspended solids or sludge concentration and comprising live bacterium or live protozoan, a nutrient material that is consumable by the live bacterium or by the live protozoan, and at least one microalgae including filamentous cyanobacteria, an illumination source configured to illuminate the mixture at least intermittently with a specified luminous flux during periods of illumination for a specified incubation period, and an agitator configured to mix the mixture under a specified shear stress during the specified incubation period, wherein the illumination of the mixture at the specified luminous flux while mixing the mixture under the specified shear stress during the incubation period incubates the mixture such that the filamentous cyanobacteria forms supporting matrices that incorporate the live bacterium or the live protozoan to provide a plurality of biologically-active bioaggregate granules.

### BRIEF DESCRIPTION OF THE DRAWINGS

The drawings illustrate generally, by way of example, but not by way of limitation, various embodiments discussed in the present document.

FIG. 1 is a schematic diagram of an example system for hydrodynamically cultivating oxygenic biologically-active bioaggregate granules.

FIG. 2 is a flow diagram of an example method of hydrodynamically cultivating oxygenic biologically-active bioaggregate granules and, optionally, using the granules for wastewater remediation or as a seed for growing additional biomass.

FIGS. 3A-3C are schematic diagrams of the example system of FIG. 1 during a portion of the example method of FIG. 2.

FIG. 4 is a cross-sectional view of an example oxygenic photogranule that can be cultivated in the example system of FIG. 1 and/or by the example method of FIG. 2.

FIGS. 5A-5C are microscopy images of an example oxygenic photogranule cultivated in the example system of FIG. 1 and/or by the example method of FIG. 2.

FIG. 6 is an autofluorescence microscopy image of filamentous cyanobacteria within an example oxygenic photogranule cultivated in the example system of FIG. 1 and/or by the example method of FIG. 2.

FIGS. 7A-7C are images of glass-jar batch reactors and paddle-blade impellers used in the experiments of EXAMPLE 1

FIG. 8 are images of sample cultivated batches after incubation under various incubation conditions in the experiments of EXAMPLE 1.

FIG. 9 is a microscopy image of an oxygenic photogranule after cultivation in the experiments of EXAMPLE 1.

FIG. 10 are light microscopic images of cultivated samples of bioaggregate granules after incubation under various incubation conditions in the experiments of EXAMPLE 1.

FIGS. 11A-11C are graphs of the particle size distributions showing the relative frequency of particles over a range of particle sizes of granules cultivated under agitation at 20 rpm in the experiments of EXAMPLE 1.

FIGS. 12A-12C are graphs of the particle size distributions showing the relative frequency of particles over a range of particle sizes of granules cultivated under agitation at 50 rpm in the experiments of EXAMPLE 1.

FIGS. 13A-13C are graphs of the particle size distributions showing the relative frequency of particles over a range of particle sizes of granules cultivated under agitation at 80 rpm in the experiments of EXAMPLE 1.

FIGS. 14A-14C are bar graphs of the five-minute sludge volume index (SVI₅) of biomass in each of the samples cultivated under various light intensities and various biomass dilution while being agitated at 20 rpm, 50 rpm, and 80 rpm, respectively in the experiments of EXAMPLE 1.

FIGS. 15A-15C are bar graphs of the thirty-minute sludge volume index (SVI₃₀) of biomass in each of the samples cultivated under various light intensities and various biomass dilutions while being agitated at 20 rpm, 50 rpm, and 80 rpm, respectively in the experiments of EXAMPLE 1.

FIGS. 16A-16C are bar graphs of the ratio of the five-minute sludge volume index relative to the thirty-minute sludge volume index (SVI₅/SVI₃₀) of biomass in each of the samples cultivated under various light intensities and various biomass dilutions while being agitated at 20 rpm, 50 rpm, and 80 rpm, respectively in the experiments of EXAMPLE 1.

FIGS. 17A-17C are bar graphs of dissolved oxygen (DO) over time for samples cultivated under various light intensities and various biomass dilutions while being agitated at 20 rpm, 50 rpm, and 80 rpm, respectively in the experiments of EXAMPLE 1.

FIGS. 18A and 18B are graphs of the evolution of the phototrophic pigments chlorophyll a and chlorophyll b, respectively, over time for samples cultivated under various light intensities and biomass dilutions while being agitated at 20 rpm in the experiments of EXAMPLE 1.

FIGS. 19A and 19B are graphs of the evolution of the phototrophic pigments chlorophyll a and chlorophyll b, respectively, over time for samples cultivated under various light intensities and biomass dilutions while being agitated at 50 rpm in the experiments of EXAMPLE 1.

FIGS. 20A and 20B are graphs of the evolution of the phototrophic pigments chlorophyll a and chlorophyll b, respectively, over time for samples cultivated under various light intensities and biomass dilutions while being agitated at 80 rpm in the experiments of EXAMPLE 1.

FIG. 21 is a photograph of a larger-scale system for cultivation of oxygenic photogranules comprising a 190 liter reaction vessel, artificial lighting, and a larger-scale agitation device as used in the experiment of EXAMPLE 2.

FIG. 22 is a photograph of oxygenic photogranules in a petri dish that resulted from the larger-scale experiment of EXAMPLE 2.

### DETAILED DESCRIPTION

The present disclosure describes systems and methods for cultivation of biologically-active bioaggregate granules comprising at least one microalgae and live bacterium or live protozoan from a water-based reaction medium, such as wastewater.

The following detailed description includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The example embodiments may be combined, other embodiments may be utilized, or structural, and logical changes may be made without departing from the scope of the present invention. While the disclosed subject matter will be described in conjunction with the enumerated claims, it will be understood that the exemplified subject matter is not intended to limit the claims to the disclosed subject matter. The following detailed description is, therefore, not to be taken in a limiting sense, and the scope of the present invention is defined by the appended claims and their equivalents.

References in the specification to “one embodiment”, “an embodiment,” “an example embodiment,” etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a concentration range of “about 0.1% to about 5%” should be interpreted to include not only the explicitly recited concentration of about 0.1 wt. % to about 5 wt. %, but also the individual concentrations (e.g., 1%, 2%, 3%, and 4%) and the sub-ranges (e.g., 0.1% to 0.5%, 1.1% to 2.2%, and 3.3% to 4.4%) within the indicated range. The statement “about X to Y” has the same meaning as “about X to about Y,”” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. Unless indicated otherwise, the statement “at least one of” when referring to a listed group is used to mean one or any combination of two or more of the members of the group. For example, the statement “at least one of A, B, and C” can have the same meaning as “A; B; C; A and B; A and C; B and C; or A, B, and C,” or the statement “at least one of D, E, F, and G” can have the same meaning as “D; E; F; G; D and E; D and F; D and G; E and F; E and G: F and G; D, E, and F; D, E, and G; D, F, and G; E, F, and G; or D, E, F, and G.” A comma can be used as a delimiter or digit group separator to the left or right of a decimal mark; for example, “0.000,1”” is equivalent to “0.0001.”

In the methods described herein, the steps can be carried out in any order without departing from the principles of the invention, except when a temporal or operational sequence is explicitly recited. Furthermore, specified steps can be carried out concurrently unless explicit language recites that they be carried out separately. For example, a recited act of doing X and a recited act of doing Y can be conducted simultaneously within a single operation, and the resulting process will fall within the literal scope of the process. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E (including with one or more steps being performed concurrent with step A or Step E), and that the sequence still falls within the literal scope of the claimed process. A given step or sub-set of steps can also be repeated.

Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, within 1%, within 0.5%, within 0.1%, within 0.05%, within 0.01%, within 0.005%, or within 0.001% of a stated value or of a stated limit of a range and includes the exact stated value or range.

The term “substantially” as used herein refers to a majority of, or mostly, such as at least about 50%, 60%, 70%, 80%, 90%, 95%, 96%, 97%, 98%, 99%, 99.5%, 99.9%, 99.99%, or at least about 99.999% or more, or 100%.

In addition, it is to be understood that the phraseology or terminology employed herein, and not otherwise defined, is for the purpose of description only and not of limitation. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

Sludge granules can include a self-mobilized microbial consortia with a relatively high density and spheroidal or generally spheroidal profiles. In an example, each granule can act as a “micro-reactor” in which biochemical transformations occur. The granules' compact structure can also withstand high-strength wastewater and shock loadings. These characteristics facilitate higher retention of biomass, giving cost and space savings compared to conventional wastewater treatment operations. In an example, microbial colonialization evolves along a spatial gradient, resulting in generic layered granular structures. These self-mobilized granular bioaggregates can, in some examples, be considered supraspecific homologs, similar in structure but differing in microbial species dominance.

In an example, the biologically-active bioaggregate granules comprise oxygenic phototrophic microalgae that produce oxygen (O₂), which can be used by bacteria or protozoa, or both, to degrade organic matter within the water-based reaction medium. For this reason, the biologically-active bioaggregate granules cultivated by the systems and methods of the present disclosure will also be referred to hereinafter as “oxygenic photogranules” or “OPGs.” In an example, the microbial community in OPGs includes filamentous cyanobacteria and algae species dominating the phototrophic outer layer where they can benefit from light exposure, while non-phototrophic bacteria dominate the inner core. The microalgae in the OPGs can then harvest CO₂ generated by the bacteria or protozoa, or both, as they consume the organic matter, and can use the harvested CO₂ for photosynthesis. In some examples, the OPGs can have a relatively large size, for example from about 0.2 millimeters (mm) to about 10 mm, which can allow for good separation of the OPGs from the water-based reaction medium in order to recover the OPGs for further wastewater treatment.

The systems and methods described herein provide for cultivation of a relatively large number of the OPGs in a relatively short period of time, after as little as 6-8 days of cultivation. This is in comparison to a prior art method, which was able to produce primarily a single OPG with a cultivation time of a few weeks. Therefore, the systems and methods described herein substantially improves the start-up time needed and the overall scale-up capability for the systems and methods of producing OPGs.

The systems and methods described herein provide for specific incubation conditions during the time period when OPGs can be formed within a wastewater. Specifically, the systems and methods include incubating a mixture of a water-based reaction medium, such as wastewater, at least one microalgae, a live bacterium or live protozoan, and a nutrient consumable by the live bacterium or by the live protozoan under the specific incubation conditions. In an example, the water-based reaction medium comprises wastewater. In an example, the water-based reaction medium comprising microbial biomass containing at least one of: microalgae, bacteria, and protozoa. In an example, the water-based reaction mixture comprising activated sludge. In an example, the specified incubation conditions include one or more of, such as two or more of, for example, three or more of, and in some examples, all of: a specified incubation period of time; at least intermittent illumination with a specified luminous flux during periods of illumination; agitating the mixture under a specified shear stress; and with a specified suspended solids concentration of an activated sludge or other microbial biomass as the source of live microalgae, bacteria, and protozoa.

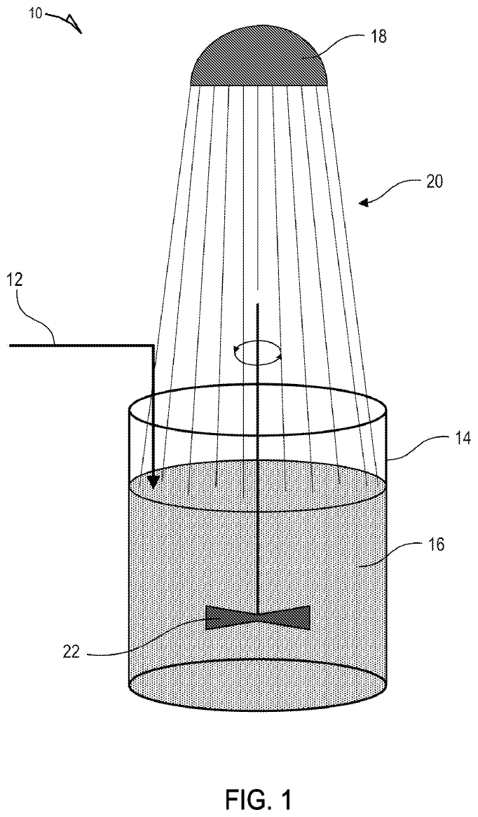

FIG. 1 is a schematic diagram of an example system 10 for cultivation of OPGs from a wastewater stream 12. The wastewater stream 12 can comprise a water-based medium, and in an example, comprises activated sludge. As used herein, the term “activated sludge” refers to a mixed liquor, a thickened mixed liquor, or biofilm present and used in water and wastewater treatment systems. Activated sludge is also sometimes referred to as “sewage sludge,” “returned activated sludge, and “waste activated sludge.” In an example, the activated sludge in the wastewater stream 12 is the inoculum source of various microorganisms that can be useful in the formation of OPGs, as described herein. The microorganisms that are present in the wastewater stream 12, for example in the activated sludge, can include, but are not limited to, one or more of: algae, cyanobacteria, bacteria, and protozoa.

The wastewater stream 12 is fed into a reaction vessel 14 where it can form at least part of a reaction mixture 16. In an example, the reaction mixture includes: the water-based medium of the wastewater stream 12; live bacterium or live protozoan, or both, which are typically present in the activated sludge, a nutrient material that is consumable by the live bacterium or the live protozoan, wherein the nutrient material can comprise organic matter in the wastewater medium and/or the activated sludge that is desired to be degraded or otherwise removed from the wastewater medium as part of a wastewater treatment; and at least one oxygenic microalgae, which may be present in the wastewater medium or in the activated sludge, or both. In some examples, all of the components desired for the formation of OPGs within the system 10 can already be present in the activated sludge of the wastewater stream 12. In other examples, one or more components can be added to the reaction mixture 16 to ensure the specified mixture of components is present in the reaction mixture 16 before incubation is begun. For example, an external source of microalgae (including cyanobacteria and/or green algae) can be added to the reaction mixture 16, additional nutrient material beyond what is already present in the wastewater stream 12 can be added to the reaction mixture 16, or additional water or water-based medium can be added.

The inventors have found that the concentration of the activated sludge in the reaction mixture 16 can affect how efficiently OPGs are formed during the incubation period. The inventors further discovered that if the activated sludge concentration is too high in the reaction mixture 16, then OPG formation is limited. Without wishing to be bound by any theory, the inventors believe that this can occur because a high concentration of activated sludge causes high turbidity in the reaction mixture 16, which can limit penetration of the light 20 into the reaction mixture 16 such that the photosynthetic microalgae, such as the filamentous cyanobacteria, do not receive a sufficient amount of light energy and, therefore, do not grow sufficiently to form the supporting matrix of the OPG. In addition, when the sludge concentration is higher, it tends to result in a thicker, more viscous reaction mixture 16, which can be harder to effectively mix and, therefore, can make it harder to expose more of the photosynthetic microalgae or filamentous cyanobacteria to the light 20. At the same time, the inventors believe that if the activated sludge concentration is too low, then there will not be a sufficient starting amount of one or more, and in some cases all of, the oxygenic microalgae, the filamentous cyanobacteria, the live bacteria or live protozoa, or the nutrients for one or more of the microalgae, the filamentous cyanobacteria, the live bacteria or the live protozoa. In an example, the specified activated sludge concentration in the reaction mixture 16 is from about 30 milligrams (mg) per L of the reaction mixture 16 to about 1,500 mg/L of the reaction mixture 16, such as from about 100 mg/L to about 700 mg/L of the reaction mixture 16, for example from about 200 mg/L to about 500 mg/L of the reaction mixture 16.

In an example, the system 10 is configured to incubate the reaction mixture 16 for a specified incubation period and under specified incubation conditions that are conducive to self-mobilization of OPGs from the microorganisms and compounds that are present in the reaction mixture 16, and in particular from those present in activated sludge within the wastewater stream 12. In an example, the specified incubation period is no more than about days, for example from about 5 days to about 10 days, such as from about 6 days to about 8 days.

The system 10 also includes an illumination source 18 that can provide at least intermittent illumination of light 20 having a specified luminous flux onto the reaction mixture 16 (described in more detail below). In the example system 10 shown in FIG. 1, the illumination source 18 is a lamp or other artificial light that is configured to generate the light having the specified luminous flux. The illumination source 18 by artificial illumination devices can be submersed in the reaction vessel 14. In other examples, the illumination source 18 can be the sun and the light 20 can be sunlight, which may or may not be altered, such as via one or more filters, one or more types of glass, or other materials through which the sunlight can be passed so that the light 20 that is incident upon the reaction mixture 16 has the specified luminous flux. In an example, the system 10 is configured so that the light will be incident on the reaction mixture 16, at least intermittently, during the specified incubation period, such that the light 20 can provide energy to drive the formation of OPGs during the incubation period. In an example, the specified illumination flux is from photosynthetic photon flux densities (PPFD) of about 30 μmol m⁻² s⁻¹ to about 1200 μmol m⁻² s⁻¹, such as from about 100 μmol m⁻² s⁻¹ to about 1100 μmol m⁻² s⁻¹, for example from about 200 μmol m⁻² s⁻¹ to about 1000 μmol m⁻² s⁻¹, such as from about 300 μmol m⁻² s⁻¹ to about 950 μmol m⁻² s⁻¹, for example from about 350 μmol m⁻² s⁻¹ to about 900 μmol m⁻² s⁻¹, such as from about 400 μmol m⁻² s⁻¹ to about 850 μmol m⁻² s⁻¹, for example from about 450 μmol m⁻² s⁻¹ to about 800 μmol m⁻² s⁻¹. If the suspended solids concentration of the reaction mixture, such as activated sludge, is higher, illumination flux should increase within this specified range. If the reactor vessel becomes deeper and the reactor volume becomes larger (described in more detail below), the illumination flux should increase within this specified range. If unaltered sunlight, which has the highest PPFD of about 2000 μmol m⁻² s⁻¹ for given peak hours of the day, is the source of light to be used and if the submersed light source emits light intensity greater than the specified range, the reactor vessel's depth and volume should be sufficiently large so that luminous flux within the vast majority of part within the reactor vessel, like greater than 95% of the vessel, is under illumination within the specified range.

The reaction vessel 14 can be chosen with a specified volume and a specified shape such that when the light 20 encounters the reaction mixture 16, it will provide sufficient light energy for the oxygenic microalgae to engage in photosynthesis such that the microalgae can multiply and grow to form part of the structure of the OPG. In the example shown in FIG. 1, the reaction vessel 14 is a cylindrical vessel, such as a lab-scale beaker or a larger industrial-scale reaction vessel. In other examples, the reaction vessel 14 can be a larger scale container for wastewater, such as a wastewater treatment pool. Those having skill in the art will appreciate that the specific volume and shape of the reaction vessel 14 can be selected based on the overall size of the system 10 desired, including a desired OPG production scale or the desired amount of wastewater to be treated. In a laboratory-scale system, the vessel 14 can have a volume as low as about 1 liter (L), such as from about 1 L to about 10 L. In a more commercial scale system, the vessel 14 can have a volume from about 100 L to about 3,000 L, such as from about 500 L to 1,500 L, for example from about 600 L to about 1,000 L. Larger volumes can also be operated with proper balance of light, shear, and the suspended solids concentration of the reaction mixture within their specified ranges.

The system 10 also includes an agitation device 22 for agitating the reaction mixture 16 during the incubation period. Prior research into the cultivation of OPGs found that it was possible to cultivate an OPG if incubation was performed under quiescent conditions, i.e., with no deliberate mechanical stirring or agitation and no deliberate imposition of thermal, compositional, or density gradients that would lead to convection or other driven fluid flow. U.S. Pat. No. 10,189,732 B2, entitled “ALGAL-SLUDGE GRANULE FOR WASTEWATER TREATMENT AND BIOENERGY FEEDSTOCK GENERATION,” issued on Jan. 29, 2019, the disclosure of which is incorporated herein by reference in its entirety, discloses systems and methods of forming one or more algal-sludge granules which are substantially similar in composition to the OPGs produced by the subject matter of the present disclosure. That patent describes that its algal-sludge granules were only found to be generated when the incubation was carried out under quiescent conditions. Previously, it was believed that if a reaction mixture was agitated during incubation, that the agitation would interfere with the formation of the granule, in effect breaking up the granulated structure before it could fully stabilize.

The inventors of the present disclosure have found that, surprisingly, it is possible to configure the system 10 to incubate OPGs even when agitating the reaction mixture 16. Without wishing to be bound by any theory, the inventors believe that agitation can act to suspend the biomass of the activated sludge and to distribute it more throughout the bulk of the reaction mixture 16, which the inventors believe exposes more of the oxygenic microalgae or filamentous cyanobacteria to the light 20, which enhances formation of the supporting matrix of the OPGs. Shear forces were also believed to increase hydrophobicity and density of the granule particles that are formed and also enhances collision of particles in the fluid media, giving the materials that can eventually form the OPGs more opportunity to come into contact and aggregate. The shear stress can also result in control over the size and shape of resulting granules, with higher shear rates tending to result in smaller and more spherically uniform aggregates. The inventors further believe that the specified shear stress of the agitation should be high enough to provide for this enhanced access to the light 20, but not so high that it begins to break down the granulation structures before they are stable enough to remain in an OPG. In an example, the specified shear stress of the agitation by the agitation device 22 is from about 0.005 Newtons per square meter (N/m²) to about 0.075 N/m², such as from about 0.01 N/m² to about 0.07 N/m².

The inventors believe that the selection pressures that promote granule formation and function suggest that granulation of OPGs occurs within a ‘goldilocks zone’ due to the interaction of chemical energies, shear pressure, and light energy in varying magnitudes. Experimental EXAMPLE 1, discussed below, examined the various energy inputs by conducting matrices of batch experiments with varying energy flows. Experimental EXAMPLE 1 shows that granulation of OPGs, and potentially of other types of granules, can occur under limited sets of environmental conditions.

In an example, the agitation is controlled and is balanced with other energy inputs into the reaction mixture 16, most notably the amount of light energy being fed into the reaction mixture 16 via the light 20 illuminated from the illumination source 18 and the amount of the relevant microorganisms in the reaction mixture 16 (e.g., the live bacterium or live protozoan, or both, and the oxygenic microalgae, as provided by the suspended solids concentration of the reaction mixture, e.g., the activated sludge concentration) as quantified by the weight concentration of activated sludge in the reaction mixture 16. For this reason, the system 10 is configured to control one or more of, such as two or more of, for example all of: the energy of the light 20 being emitted onto the reaction mixture 16 by ensuring the light 20 has a specified luminous flux during the incubation period; the agitation energy supplied to the reaction mixture 16 by controlling the agitation device 22 to a specified shear stress during the incubation period; and the potential chemical energy in the reaction mixture 16 by controlling the concentration of the activated sludge to a specified suspended solids or sludge concentration within the reaction mixture 16.

Each of the specified process parameters can depend on one another such that changing the magnitude of one of the parameters selected, affects the required magnitude of the other parameters. For example, if the activated sludge concentration is on the higher end of the specified suspended solids or sludge concentration range, then it may be compensated for by increasing the luminous flux so that the photosynthetic microalgae are exposed to higher energy light 20, which can increase the rate of microalgae and/or filamentous cyanobacteria growth. In another example, if the activated sludge concentration is on the higher end of the range, then the shear stress can be increased to more effectively dissipate the greater quantity of activated sludge within the reaction mixture 16 and expose more of the sludge and its components to the light 20. Those having skill in the art will be able to appreciate balancing each of the particular parameters that the inventors have found to be most important in OPG formation: that is, the activated sludge concentration in the reaction mixture 16, the luminous flux of the light 20 that is emitted onto the reaction mixture 16, and the shear stress at which the reaction mixture 16 is agitated by the agitation device 22.

Each of the specified process parameters can depend on other parameters of the system 10, such as the volume of the reaction vessel 14. For example, if the vessel 14 is a smaller, laboratory-scale reactor (i.e., about 1 L), then the specified shear stress range is narrower and cannot go as high, e.g., from about 0.01 N/m² to about 0.04 N/m², because with such a small vessel, larger shear stresses will more readily break up the OPGs as they are formed. But, for a large-scale vessel 14, such as a 600 L vessel, a wider range of shear stresses and higher shear stresses, can be used, e.g., from about 0.01 N/m² to about 0.07 N/m². Similarly, for a larger volume vessel, a more powerful luminous flux may be acceptable because there is a larger volume to absorb the extra energy.

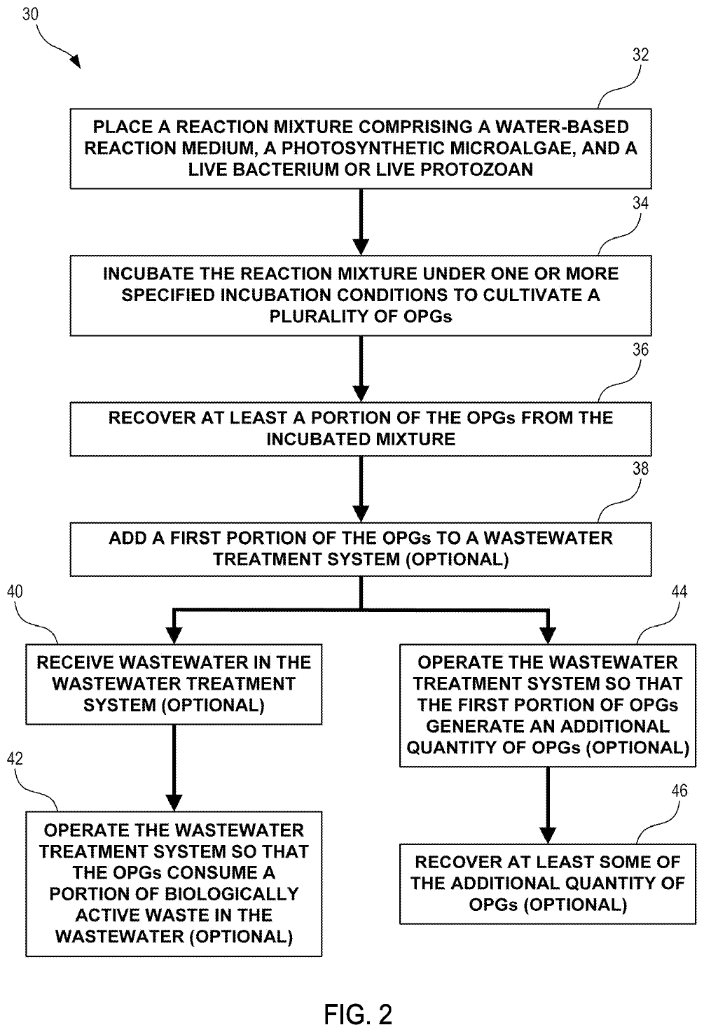

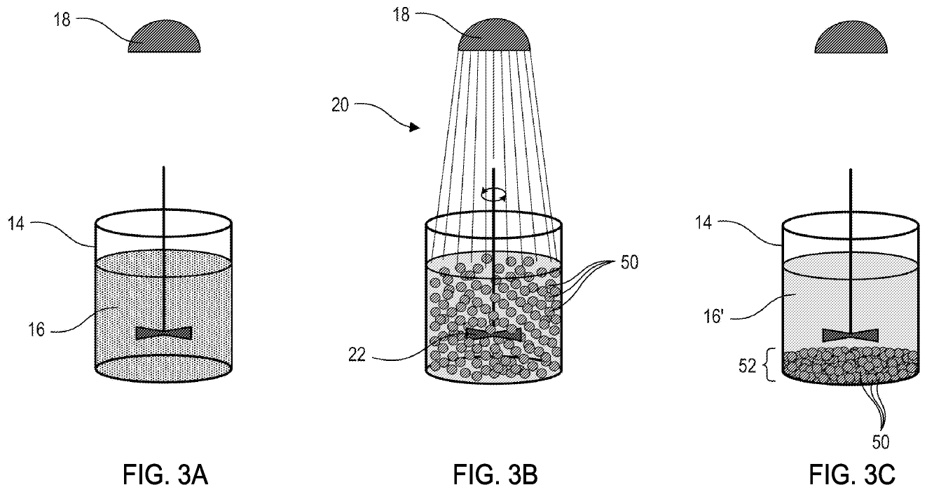

FIG. 2 is a flow diagram of an example method 30 of cultivating OPGs. FIGS. 3A, 3B, and 3C show a visual representation of some of the steps of the method 30 of FIG. 2 if it were performed using the example system 10 of FIG. 1. The method 30 can include, at step 32, placing a reaction mixture in a vessel, such as the reaction mixture 16 in the reaction vessel 14 as shown in the example of FIG. 3A. In an example, the reaction mixture comprises a water-based reaction medium and at least one photosynthetic microalgae, such as a microalgae including filamentous cyanobacteria. The water-based reaction medium can also include a nutrient material that is consumable by a live bacterium or by a live protozoan present in the water-based reaction medium. In an example, placing the reaction mixture in the vessel (step 32) can include adding a wastewater stream that comprises activated sludge. In some examples, placing the reaction mixture in the vessel (step 32) can include placing the wastewater stream comprising the activated sludge into the vessel and modifying the concentration of the activated sludge to a specified activated sludge concentration, for example by adding water or a water-based solution to the vessel to dilute the activated sludge from its initial concentration to the specified suspended solids or sludge concentration. As described above, in an example, the specified sludge concentration is from about 30 milligrams (mg) per L of the reaction mixture to about 1,500 mg/L of the reaction mixture, such as from about 100 mg/L to about 700 mg/L of the reaction mixture, for example from about 200 mg/L to about 500 mg/L of the reaction mixtures.

After placing the reaction mixture in the vessel (step 32), the method 30 can include, at step 34, incubating the reaction mixture for a specified incubation period under one or more specified incubation conditions, such as two or more of the specified incubation conditions, and in some examples all three of the specified incubation conditions. As described above, in an example, the specified incubation period is no more than about 15 days and dependent on the size of reactor and light intensity, for example from about 5 days to about 10 days, such as from about 3 days to about 12 days, for example from about 6 days to about 8 days. In an example, the specified incubation conditions can include one or more of, for example two or more of, and in some examples, all of: at least intermittent illumination with a specified luminous flux during periods of illumination for the specified incubation period; agitating the reaction mixture at a specified shear stress for the specified incubation period; and a specified suspended solids concentration of the reaction mixture. As described above, in an example, the specified illumination flux is from about 30 mol m's⁻¹ to about 1200 μmol m⁻² s⁻¹, such as from about 100 μmol m⁻² s⁻¹ to about 1100 μmol m⁻² s⁻¹, for example from about 200 μmol m⁻² s⁻¹ to about 1000 μmol m⁻² s⁻¹, such as from about 300 μmol m⁻² s⁻¹ to about 950 μmol m⁻² s⁻¹, for example from about 350 μmol m⁻² s⁻¹ to about 900 μmol m⁻² s⁻¹, such as from about 400 μmol m⁻² s⁻¹ to about 850 μmol m⁻² s⁻¹, for example from about 450 μmol m⁻² s⁻¹ to about 800 μmol m⁻² s⁻¹. As is also described above, in an example, the specified shear stress can be from about 0.005 N/m² to about 0.075 N/m², such as from about 0.01 N/m² to about 0.07 N/m². As is also described above, in an example, the specified solids concentration in the reaction mixture is from about 30 milligrams (mg) per L of the reaction mixture to about 1,500 mg/L of the reaction mixture, such as from about 100 mg/L to about 700 mg/L of the reaction mixture, for example from about 200 mg/L to about 500 mg/L of the reaction mixture.

In an example, during the incubation of step 34, one or more of the photosynthetic microalgae, such as the filamentous cyanobacteria, forms a supporting matrix that incorporates the live bacterium or live protozoan into a biologically-active bioaggregate granule, e.g., in the form of one or more OPGs. In an example, the incubation of step 34 produces a plurality of the OPGs. FIG. 3B shows an example of a plurality of OPGs 50 that have formed in the vessel 14 as a result of the incubation of step 34, including illumination of light 20 with the specified luminous flux by the illumination source 18 and agitation at the specified shear stress with the agitation device 22.

After the incubation (step 34), the method 30 can include, at step 36, recovering at least a portion of the plurality of OPGs from the incubated mixture. Recovery of a plurality of the OPGs (step 34) can include separating the OPGs from the incubated reaction mixture. In an example, separation of the OPGs from the incubated reaction mixture can include settling the OPGs or allowing the OPGs to settle, which typically occurs in 10 minutes or less after agitation is ceased, and then removing at least a portion of the incubated reaction mixture from the OPGs. FIG. 3C shows a conceptual depiction of the OPGs 50 after they have been allowed to settle to the bottom of the vessel 14, resulting in a relatively dense settled mass 52 of the OPGs 50. After the settling depicted in FIG. 3C has occurred, the incubated reaction mixture 16′ can be removed from the vessel 14, such as by siphoning or pumping the incubated reaction mixture 16′ out of the vessel 14 (not shown in FIG. 3C).

The recovery of the OPGs (step 36) can allow the OPGs to be used for other processing, such as in a method of wastewater remediation. For example, the method 30 can optionally include after recovering the OPGs (step 36), at step 38, adding a first portion of the OPGs into a wastewater treatment system. Then, the method 30 can optionally include, at step 40, receiving wastewater into the wastewater treatment system, wherein the wastewater has a first amount of a biologically-active waste per unit volume. Next, the method 30 can include, at step 42, operating the wastewater treatment system under operating conditions that allow said first portion of the OPGs to consume a portion of the biologically-active waste to provide a processed wastewater having a second amount of biologically-active waste per unit volume that is lower than the first amount.

In another example, after adding a first portion of the OPGs into a wastewater treatment system (step 38), the method 30 can optionally include, at step 44, operating said wastewater treatment system under operating conditions that allow the first portion of the OPGs to generate an additional quantity of OPGs that is more than the first portion. Then, the method 30 can include, at step 46, recovering from at least some of the additional quantity of the OPGs from the wastewater treatment system, while still leaving in a sufficient amount of OPGs in the wastewater treatment system to continue operation of the wastewater treatment system for wastewater remediation.

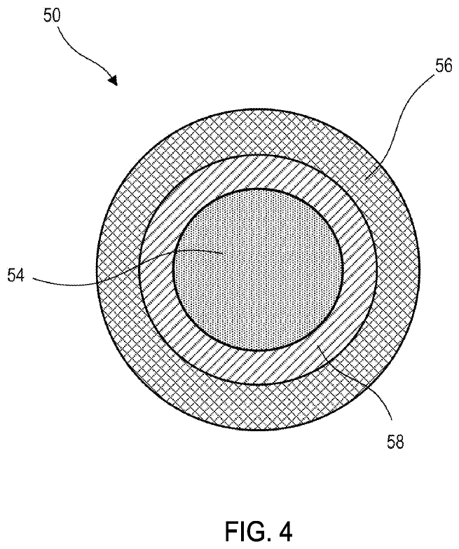

In an example, the operation of the system 10 or performing the method 30 at the specified incubation conditions (e.g., one or more of, such as two or more of, and in some examples all three of: the specified luminous flux for the light 20; the specified shear stress produced by the agitation device 22; and the specified activated sludge concentration within the reaction mixture 16 during incubation) can result in OPGs having a certain structural configuration. FIG. 4 shows a conceptual view of the cross section of an example OPG 50 formed by the system 10 and method 30 of the present disclosure. Those having skill in the art will appreciate that the structure shown in FIG. 4 is meant only as a conceptual illustration of the structure that the inventors believe occurs for one example OPG 50. The specific structure of the OPG 50 shown in FIG. 4 is not to be taken as limiting.

In an example, the OPG 50 includes an inner core 54, an outer layer 56, and a middle layer 58 located between the inner core 52 and the outer layer 54. In an example, the outer layer 56 of the OPG 50 that form under the specified incubation conditions comprise a supporting matrix formed from filamentous cyanobacteria, and in particular motile filamentous cyanobacteria. In an example, the middle layer 58 can comprise some of the live bacteria or live protozoa, or both, and in some examples, green algae and a smaller concentration of filamentous cyanobacteria compared to that in the outer layer 56. In an example, the inner core 54 primarily comprises sludge-like material, e.g., a small amount of the activated sludge. The structure of the OPG 50 has been found to not only provide for a stable structure during the formation of the OPG 50, but it can also allow the fully-formed OPGs 50 to withstand larger forces, including shock loading of wastewater or more vigorous agitation during wastewater treatment, and thus can facilitate a relatively higher retention of biomass that can be used for subsequent wastewater treatment.

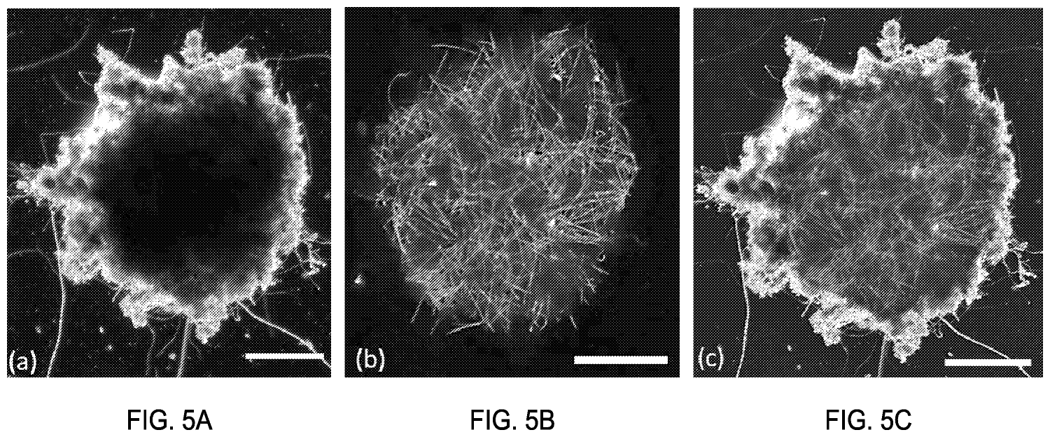

FIGS. 5A-5C shows microscopy images of example OPGs formed by the system 10 and the method 30 described herein. FIG. 5A is a brightfield (BF) microscopy image of the example OPG. FIG. 5B is an autofluorescence (AF) microscopy image of the example OPG. FIG. 5C is an image with the AF image superimposed onto the BF image. The scale bar shown in each of FIGS. 5A-5C is equal to 500 micrometers (μm).

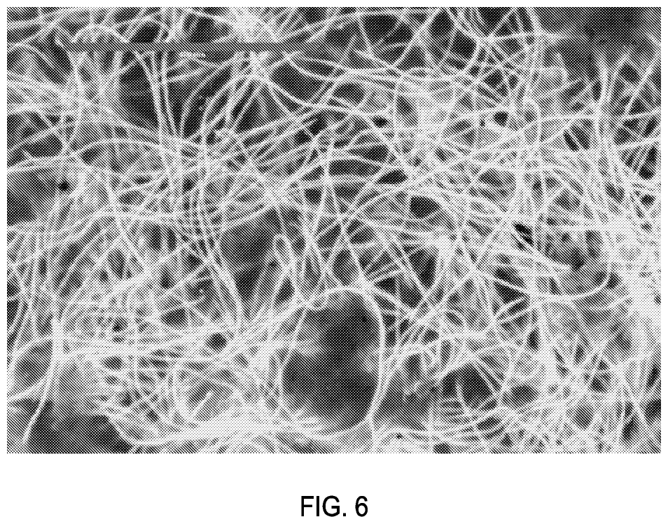

In an example, it was found that substantial growth of filamentous cyanobacteria such as genus Microcoleus, Phormidesmis, Oscillatoria, Letptolyngbia, Plectonema, Geitlerinema, Tychonema, Pseudanabaena filamentous cyanobacteria, which all belong to the order Oscillatoriales, can be particular helpful in granulation. It is believed that this occurs because growth of the filamentous cyanobacteria, in particular Oscillatoriales, in a relatively high cell density within activated sludge allows the gliding motility of the motile filamentous cyanobacteria to form interwoven structures that can act as a supporting matrix for the other components of the OPG, such as the activated sludge and the live bacteria or live protozoa, or both. FIG. 6 is phycobilin autofluorescence microscopy image of filamentous cyanobacteria in the outer layer 56 of the example OPG of FIG. 4. The scale bar in FIG. 6 is equal to 400 μm.

In an example, the addition of a small amount of calcium ions (Ca²⁺) can enhance granulation. It is believed that this occurred because extracellular proteins that are involved in cell motility of many filamentous cyanobacteria are Ca²⁺-dependent proteins, which results in enhanced motility and substantial growth of the filamentous cyanobacteria supporting matrix.

Further details regarding the OPGs and the systems and methods described herein are provided in: Gikonyo, J. G., et al., “In vivo evaluation of oxygenic photogranules' photosynthetic capacity by pulse amplitude modulation and phototrophic-irradiance curves,” ACS ES&T Engineering 1(3), 551-61 (2021); Park, C. et al., “Unmasking photogranulation in decreasing glacial albedo and net autotrophic wastewater treatment,” Environmental Microbiology, (2021), available at https://doi.org/10.1111/1462-2920.15780; Gikonyo, J. G. et al., “Hydrodynamic granulation of oxygenic photogranules,” Environmental Science: Water Research & Technology 7, 427-40 (2021); and Park et al., U.S. Pat. No. 10,189,732, entitled “ALGAL-SLUDGE GRANULE FOR WASTEWATER TREATMENT AND BIOENERGY FEEDSTOCK GENERATION,” issued on Jan. 29, 2019; the disclosures of which are incorporated by reference herein in their entireties.

### EXAMPLES

Various embodiments of the present invention can be better understood by reference to the following EXAMPLES which are offered by way of illustration. The present invention is not limited to the EXAMPLES given herein.

### Example 1

### Experimental Setup

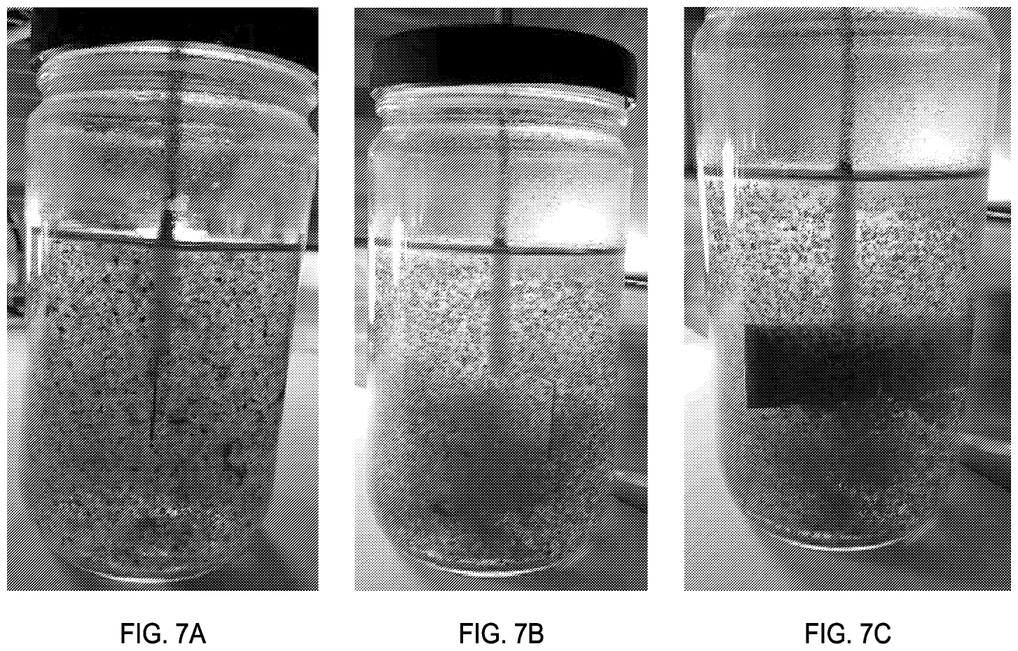

A jar-test rig mixer was used to induce mixing in batch reactors. The mixer's variable speed drives were calibrated to run at speeds of 20 rpm, 50 rpm, and 80 rpm. The paddle-blade impellers had a diameter of 5 centimeters (cm), a width 2.9 cm, and were set at a clearance of 5 cm from the vessel bottom. Clear cylindrical-glass jars having a volume of 1 L were used for the experiment with an operating volume of 800 mL. FIGS. 7A-7C are images of the glass-jar batch reactors and the paddle-blade impellers used in EXAMPLE 1. The 20 rpm, 50 rpm, and 80 rpm mixing speeds induced theoretical shear stresses of 0.01 N/m² (11 s⁻¹), 0.04 N/m² (39 s⁻¹), and 0.07 N/m² (73 s⁻¹), respectively. Batches were operated under three light intensities of 6.4±1 KLux, 12.7±1 KLux, and 25±1 KLux, using 9 W LEDs (EcoSmart, daylight 5000 K) with a luminosity of 840 Lumens. These light conditions were calculated as roughly equivalent to photosynthetic photon flux densities of 117, 216, and 450 μmol m⁻² s⁻¹, respectively, and were provided continuously for a duration of 8 days. There was no supplemental aeration in all batch systems.

Reactor Seeding

Activated-sludge inoculum was collected from a local wastewater treatment plant on three different days. The collected activated sludge had mixed-liquor suspended solids of 3,900-5,300 mg/L. This inoculum was diluted with deionized water giving ×4, ×2 and ×1 dilution inoculum. The batch reactors were then seeded and capped to minimize evaporation. Each batch ensemble was set up with a constant mixing speed with different light intensities and dilution (e.g., 9 batches for 80 rpm ensemble combined with three light conditions and three dilutions). There was a total of 27 batches, which were each operated in duplicate, were operated. The properties of each batch are provided in Table 1.

TABLE 1
Experimental set-up with combinations of different
conditions of light, mixing, and biomass dilution

| Light | 20 rpm | 50 rpm | 80 rpm |
|---|---|---|---|
| Mixing | (0.01N m⁻²) | (0.04N m⁻²) | (0.07N m⁻²) |
| 6.4 KLux | x4, x2, and x1 | x4, x2, and x1 | x4, x2, and x1 |
| (117 μmol m⁻² s⁻¹) | dilution | dilution | dilution |
| 12.7 KLux | x4, x2, and x1 | x4, x2, and x1 | x4, x2, and x1 |
| (216 μmol m⁻² s⁻¹) | dilution | dilution | dilution |
| 25 KLux | x4, x2, and x1 | x4, x2, and x1 | x4, x2, and x1 |
| (450 μmol m⁻² s⁻¹) | dilution | dilution | dilution |

Analytical Methods

The sludge volume index (SVI) was determined after 5 minutes of biomass settling (SVI₅) and after 30 minutes of biomass settling (SVI₃₀) based on Standard Methods (2710D). The total and volatile suspended solids (TSS and VSS), chlorophyll pigments, and dissolved oxygen (DO) were either measured or determined following a designated method in Standard Methods.

Imaging Analysis and Microscopy

5 milliliter (mL) samples were periodically collected during the course of the experiment and were placed in Petri dishes so that high-resolution images could be captured. The particle size and number of particles were determined using IMAGE-PRO v10 (Media Cybernetics, Inc., Rockville, MD, USA). A Weibull distribution was used to describe the particle size distribution (PSD). Light microscopy (EVOS FL Color AMEFC-4300) was conducted using bright field and epifluorescence (RFP light cube-532 excitation/590 Emission) to characterize changing morphology and microbial composition. Enrichment of cyanobacteria expected with OPG granulation results in golden-orange fluorescence due to the cyanobacteria's phycobiliproteins, specifically phycoerythrin.

Statistical Analysis

MINITAB v.17 (Minitab, LLC, State College, PA, USA) and EXCEL 2010 (Microsoft Corp., Redmond, WA, USA) were applied for all statistical analysis. The significance of the results was determined at the 0.05 probability level. A metric ratio of the SVI₅ relative to the SVI₃₀ (SVI₅/SVI₃₀) was used to describe temporal settleability of samples. Multiple regression models (least squares) were fit to the change in SVI₅/SVI₃₀ ratio data for day 6 and day 8 using the experimental parameters (light, mixing, and dilution) as predictor variables. Models were developed without (M) and with (Mⁱ) parameter interactions. Pearson correlation was used to evaluate the correlation between variables.

### Results

Generation of OPGs by Hydrodynamic Batches Using Activated-Sludge Inoculum

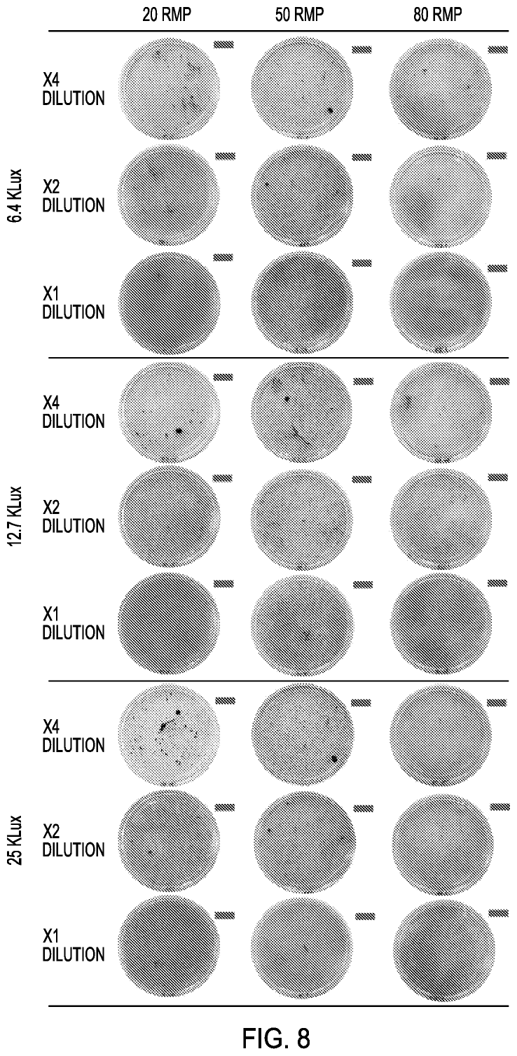

Granular aggregates appeared in several cultivation batches operated with different magnitudes of mixing, light, and inoculum concentration. Images of the 5 mL sample collected on Day 8 for each of the 27 sample cultivation batches is shown in FIG. 8. The images show that batches with lower mixing speeds (20 rpm and 50 rpm) and higher biomass dilutions (×4 and ×2) across all three light conditions yielded granular aggregates. For the 80 rpm mixing batches, primarily one set (×4 dilution and 6.4 KLux) was easily discernable for granule formation. All batches conducted with undiluted (xl) inoculum did not reveal identifiable granules, regardless of any mixing and light conditions provided.

Light microscopy confirmed that granules formed in these hydrodynamic batches were related to OPGs cultivated under hydrostatic conditions or those produced in reactor operation seeded with hydrostatically-formed OPGs, as shown in FIGS. 5, 6, 9, and 10. Like previously reported OPGs, the granules' outer surface was dominated by filamentous cyanobacteria that formed an interwoven mat-like structure. Autofluorescence microscopy also led to clear visualization of these filamentous cyanobacteria due to their phycobilin pigment (FIG. 6). The flexing and gliding motility of filamentous cyanobacteria was observed, indicating that they belong to the subsection III cyanobacteria, which are different from other filamentous cyanobacteria (i.e., subsections IV-V) that are non-motile and also undergo cell differentiation. The enrichment of the subsection III cyanobacteria, or “Oscillatoriales” in the traditional sense, is well documented from OPGs cultivated under hydrostatic conditions and reactor operation.

Evolution of Particle Sizes

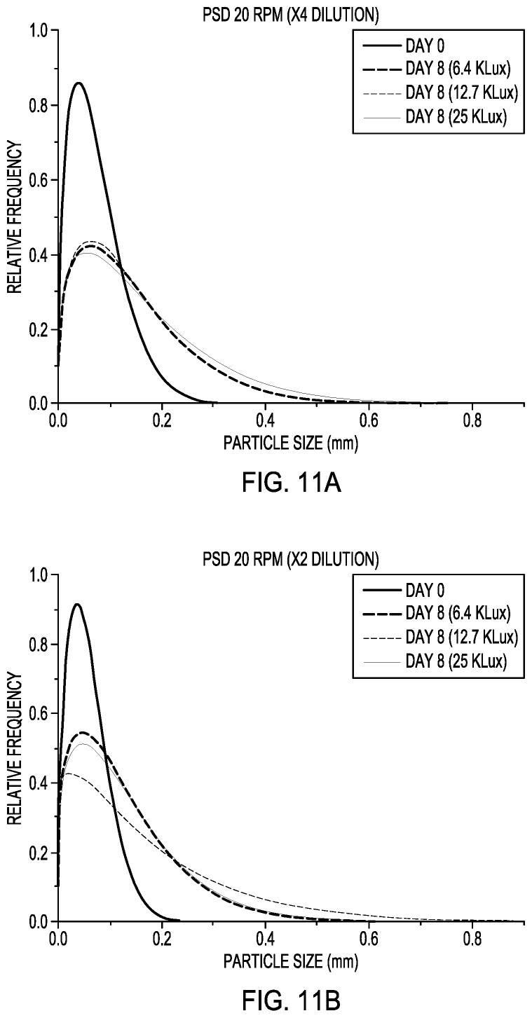

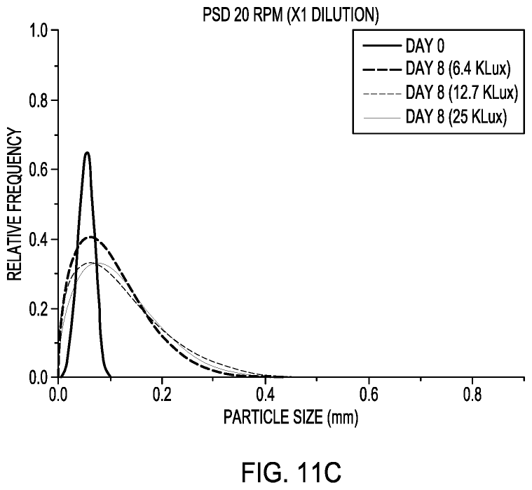

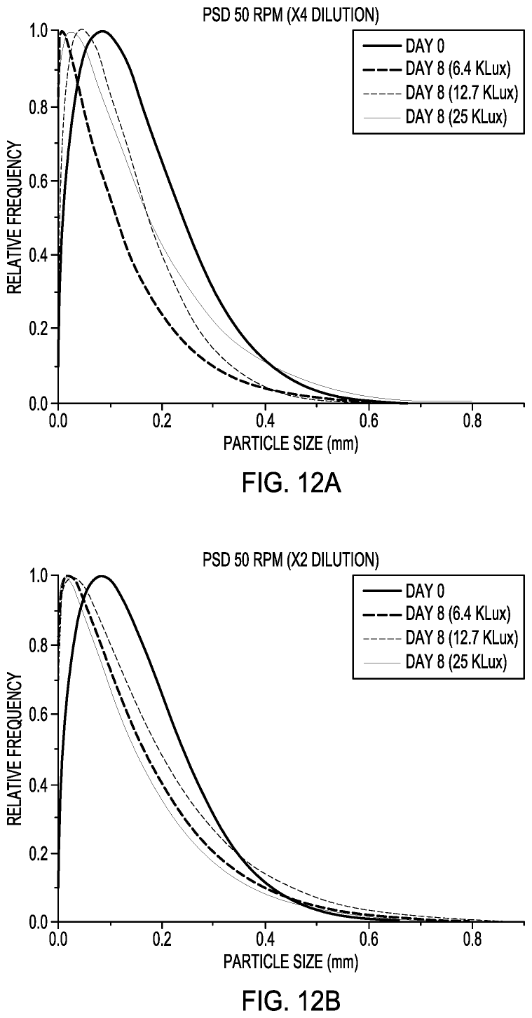

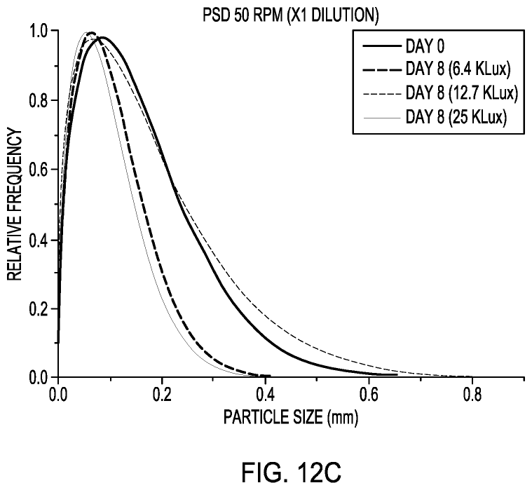

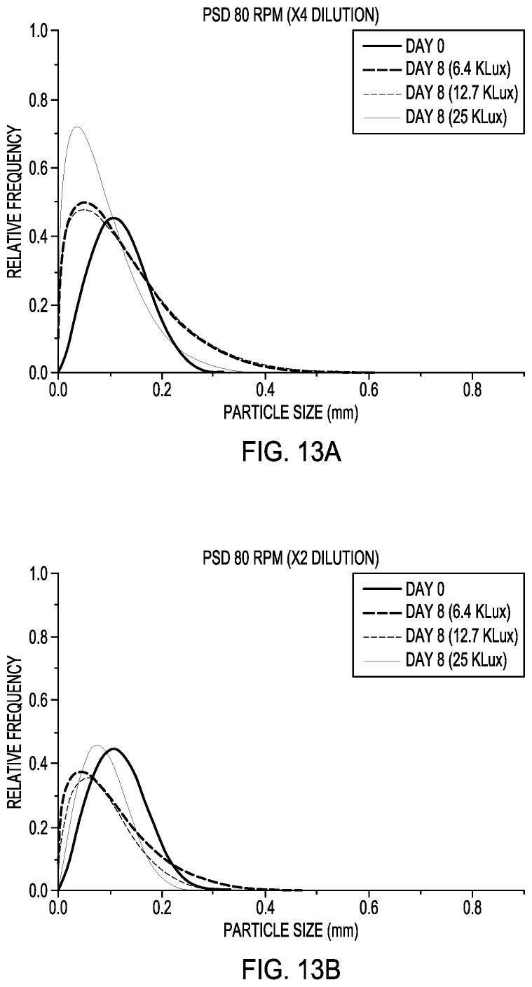

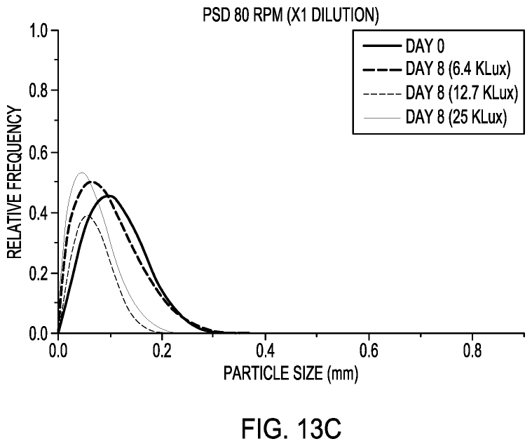

Particle size changes that occurred in the hydrodynamic batches was analyzed. Table 2 provides a statistical summary of the size distribution characteristics for each of the batches. FIGS. 11, 12, and 13 show graphical depictions of the particle size distribution (Weibull distribution) showing the relative frequency of particles over a range of particle sizes. FIGS. 11A, 11B, and 11C show the particle size distributions for the 20 rpm batches, FIGS. 12A, 12B, and 12C show the particle size distributions for the 50 rpm batches, and FIGS. 13A, 13B, and 13C shows the particle size distribution for the 80 rpm batches.

TABLE 2
Statistical summary of particle size distribution characteristics from 27 batches

| Mixing | Dilution | | | Mean | Standard | Mode | Median |
|---|---|---|---|---|---|---|---|
| (rpm) | factor | | N | (mm) | deviation | (mm) | (mm) |
| 20 | 4 | Day 0 | 2727 | 0.08 | 0.06 | 0.04 | 0.16 |
| | | Day 8 (6.4 KLux) | 2134 | 0.15 | 0.13 | 0.04 | 0.22 |
| | | Day 8 (12.7 KLux) | 6502 | 0.14 | 0.13 | 0.04 | 0.22 |
| | | Day 8 (25 KLux) | 2132 | 0.16 | 0.16 | 0.04 | 0.23 |
| | 2 | Day 0 | 59 | 0.06 | 0.05 | 0.04 | 0.15 |
| | | Day 8 (6.4 KLux) | 2820 | 0.13 | 0.13 | 0.05 | 0.19 |
| | | Day 8 (12.7 KLux) | 3996 | 0.18 | 0.23 | 0.05 | 0.22 |
| | | Day 8 (25 KLux) | 2820 | 0.14 | 0.14 | 0.05 | 0.20 |
| | 1 | Day 0 | 19 | 0.06 | 0.03 | 0.04 | 0.15 |
| | | Day 8 (6.4 KLux) | 723 | 0.11 | 0.08 | 0.04 | 0.18 |
| | | Day 8 (12.7 KLux) | 859 | 0.13 | 0.11 | 0.05 | 0.20 |
| | | Day 8 (25 KLux) | 1255 | 0.12 | 0.10 | 0.05 | 0.20 |
| 50 | 4 | Day 0 | 2446 | 0.16 | 0.14 | 0.06 | 0.24 |
| | | Day 8 (6.4 KLux) | 9032 | 0.11 | 0.09 | 0.04 | 0.19 |
| | | Day 8 (12.7 KLux) | 8779 | 0.15 | 0.14 | 0.04 | 0.19 |
| | | Day 8 (25 KLux) | 9183 | 0.15 | 0.11 | 0.04 | 0.23 |
| | 2 | Day 0 | 296 | 0.13 | 0.08 | 0.06 | 0.24 |
| | | Day 8 (6.4 KLux) | 8779 | 0.15 | 0.14 | 0.04 | 0.22 |
| | | Day 8 (12.7 KLux) | 7310 | 0.16 | 0.15 | 0.04 | 0.23 |
| | | Day 8 (25 KLux) | 9161 | 0.14 | 0.14 | 0.04 | 0.21 |
| | 1 | Day 0 | 42 | 0.11 | 0.06 | 0.06 | 0.21 |
| | | Day 8 (6.4 KLux) | 102 | 0.11 | 0.08 | 0.04 | 0.19 |
| | | Day 8 (12.7 KLux) | 37 | 0.09 | 0.08 | 0.05 | 0.17 |
| | | Day 8 (25 KLux) | 172 | 0.10 | 0.08 | 0.04 | 0.18 |
| 80 | 4 | Day 0 | 285 | 0.12 | 0.05 | 0.06 | 0.24 |
| | | Day 8 (6.4 KLux) | 4096 | 0.13 | 0.11 | 0.04 | 0.20 |
| | | Day 8 (12.7 KLux) | 5125 | 0.14 | 0.12 | 0.04 | 0.21 |
| | | Day 8 (25 KLux) | 1303 | 0.09 | 0.08 | 0.04 | 0.17 |
| | 2 | Day 0 | 285 | 0.12 | 0.05 | 0.06 | 0.22 |
| | | Day 8 (6.4 KLux) | 1899 | 0.11 | 0.10 | 0.04 | 0.18 |
| | | Day 8 (12.7 KLux) | 322 | 0.09 | 0.07 | 0.04 | 0.18 |
| | | Day 8 (25 KLux) | 170 | 0.09 | 0.05 | 0.04 | 0.18 |
| | 1 | Day 0 | 42 | 0.11 | 0.06 | 0.06 | 0.21 |
| | | Day 8 (6.4 KLux) | 185 | 0.10 | 0.07 | 0.04 | 0.18 |
| | | Day 8 (12.7 KLux) | 26 | 0.07 | 0.04 | 0.04 | 0.17 |
| | | Day 8 (25 KLux) | 17 | 0.07 | 0.05 | 0.04 | 0.16 |

The data shows that an increase of the consortia particle concentration around the mean size was observed under all conditions with 20 rpm agitation (FIGS. 11A-11C; Table 2). The mean particle sizes for the ×4 and the ×2 dilution ensembles, most of which showed the formation of OPGs (FIG. 8), changed from an average of 0.08 mm and 0.06 mm both to 0.15 (±0.004) mm, while the mean size of the undiluted ensemble increased from an average of 0.06 mm to 0.11 (±0.004) mm. The increase in mean particle size was also accompanied by positively skewed distributions.

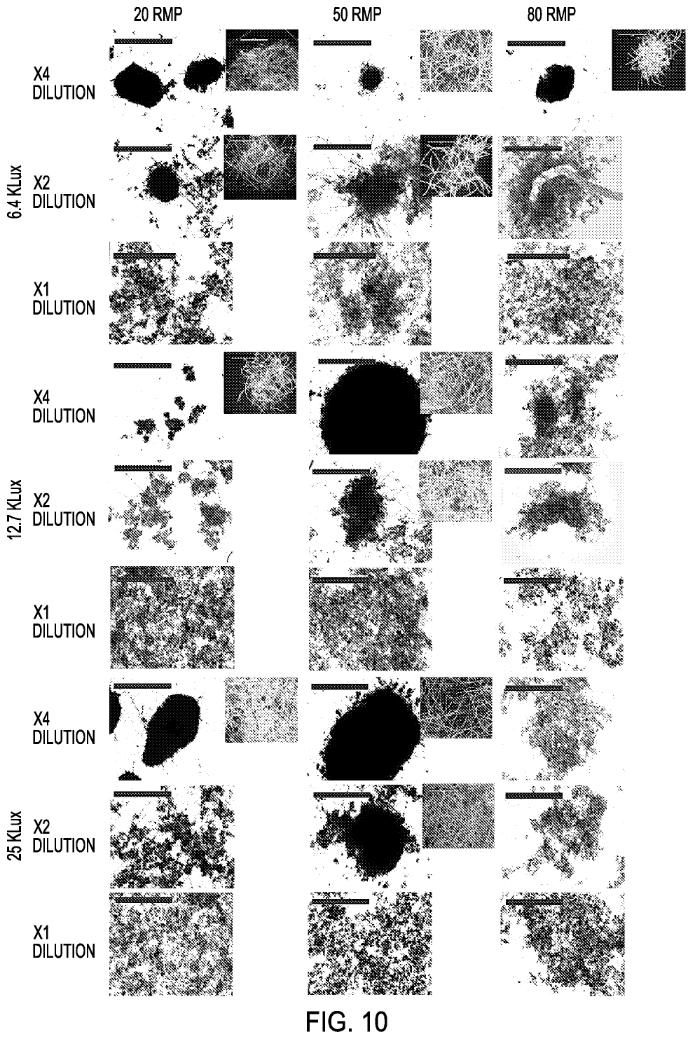

For the 50 rpm sets (FIGS. 12A-12C; Table 2), the mean particle size exhibited decreases for the ×4 and the xl dilutions and an increase for the ×2 dilution. For the ×4 dilution ensemble, the mean particle size decreased from 0.16 mm to 0.14 (±0.019) mm. The mean particle size for the ×2 dilutions increased from 0.13 mm to 0.15 (±0.008) mm, while those of the undiluted samples decreased from 0.11 mm to 0.10 (±0.01) mm. While the particle size distributions indicated to shift towards smaller sizes for all of the 50 rpm batches, the sets with the ×4 and the ×2 dilutions are where the OPGs appeared to exhibit more significant positive skews. The inventors believe that the overall decrease in both mean and median size for the 50 rpm ensemble can be attributed to particle breakage and detachment resulting from higher mixing-induced particle-particle collisions. However, the positive skews observed in the higher-dilution sets indicate an increase in the concentration of larger particle sizes, including granules (FIG. 8; FIG. 10). This increase in sizes for the 50 rpm samples suggests a microbially driven aggregation withstanding the shear limitations.

The 80 rpm samples had an average initial mean particle size of 0.12 (±0.002) mm. This mean was conserved at 0.12 (±0.026) mm for the ×4 dilution sets while decreasing to 0.10 (±0.008) mm and 0.08 (±0.018) mm for the ×2 and the xl dilutions, respectively. Compared to day 0 samples, most 80 rpm sets experienced a shift toward a smaller particle size distribution. In addition, the particle size distribution became significantly positively skewed for the ×4 dilution samples having the two lower light conditions, indicating an increase in the concentration of larger particle sizes.

Assessment of Settleability with SVI

SVI was used to assess the ease of solids separation in wastewater treatment. Activated sludge with effective settling typically shows SVI₃₀ of less than 150 mL/g, while the inventors have found that OPGs formed by the systems and methods described herein have average SVI₃₀ of around 53 mL/g. In addition, aerobic granules have a reported typical SVI₅ of around 88 mL/g and algal-bacterial granules have a reported typical SVI₅ of around 48 mL/g.

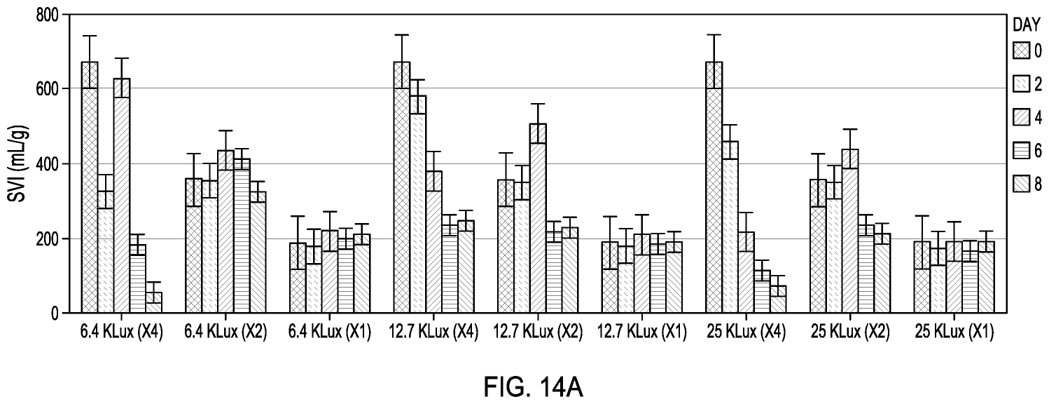

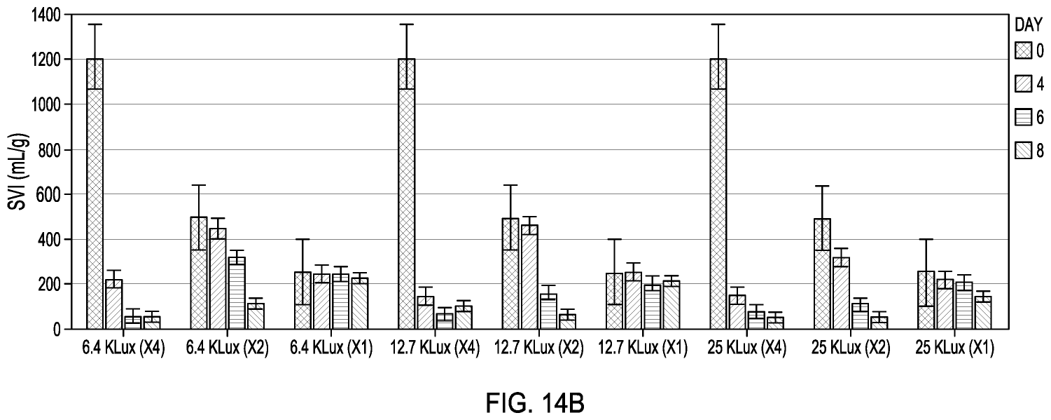

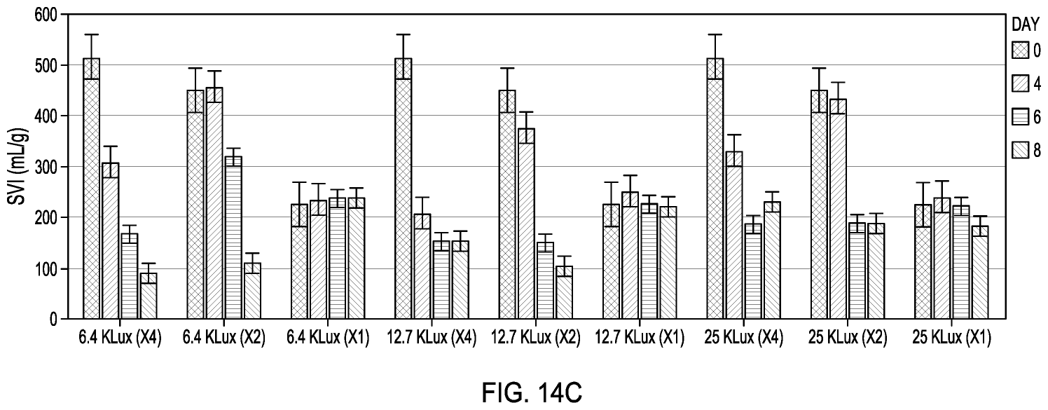

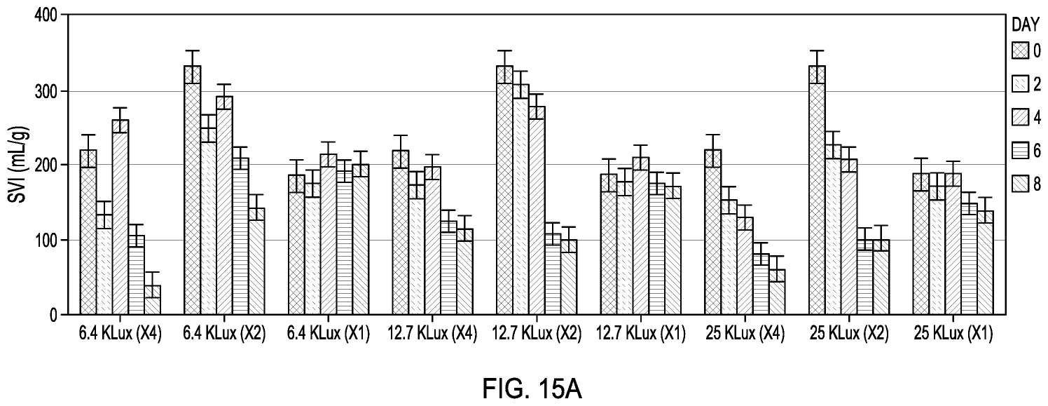

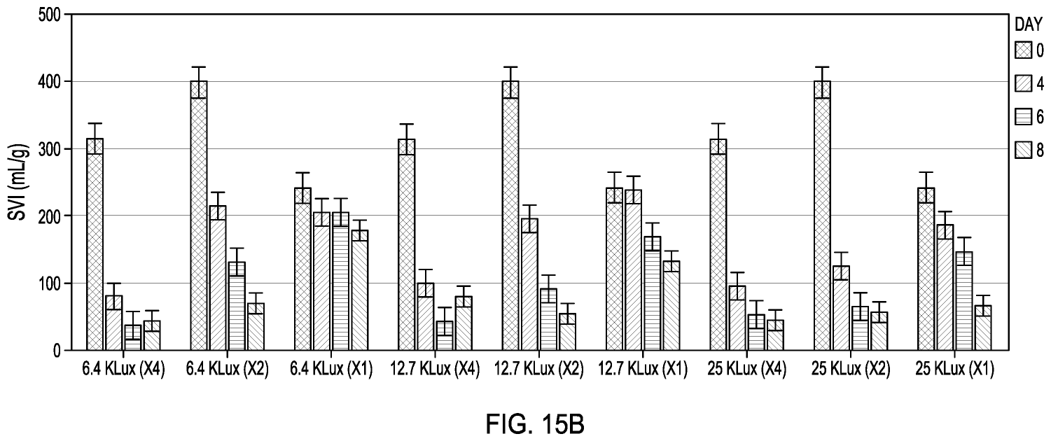

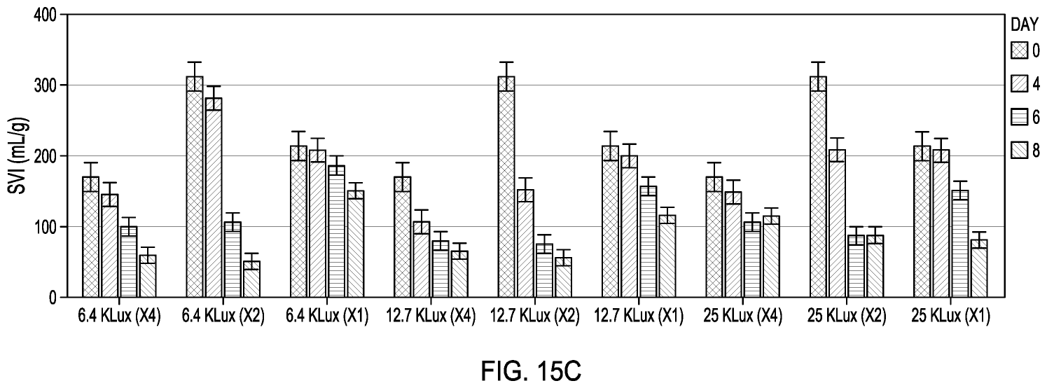

FIGS. 14A, 14B, and 14C show the SVI 5 for the 20 rpm samples, the 50 rpm samples, and the 80 rpm samples, respectively, measured after 0 days, 2 days, 4, days, 6 days, and 8 days of incubation. FIGS. 15A, 15B, and 15C show the SVI₃₀ for the 20 rpm samples, the 50 rpm samples, and the 80 rpm samples, respectively, measured after 0 days, 2 days, 4, days, 6 days, and 8 days of incubation.

The undiluted activated-sludge inoculum had an average SVI₅ of 221 mL/g and an average SVI₃₀ of 219 mL/g. Activated-sludge inoculum with ×4 and ×2 dilutions showed average SVI₅ values of 798 mL/g and 432 mL/g and SVI₃₀ of 235 mL/g and 246 mL/g, respectively. The significant increase of SVI₅ with dilution indicates poor settleability reflecting dilution-induced reduction of inter-particle interaction that diminishes flocculent (Type II) and hindered (Type III) settling effects in activated sludge.

In 20 rpm and 50 rpm batches, the ×4 dilution sets, which all clearly showed the formation of OPGs (see FIG. 8), exhibited clear decline in SVI₅ over the batch period. Except for one set, their terminal SVI₅, 65±5 mL/g, was comparable to that of aerobic granules and algal-bacterial granules. The incongruent 20 rpm and 12.7 KLux-×4 batch, which clearly produced OPGs, had SVI₅ of 245 mL/g. The SVI result indicates that the formation of granules in this set was not sufficient to lower SVI₅ for bulk biomass. This statement may also apply to the sets with 20 rpm and ×2 dilutions under all light conditions, although granule formation was less conspicuous than the former batch. In contrast, batches with 50 rpm and ×2 dilution showed SVI₅ proximate to or much less than 100 mL/g, indicating that these sets overall resulted in effectively settling biomass, including granules. The undiluted sets in 20 rpm and 50 rpm had little or marginal change in SVI₅ as well as high terminal SVI₅ values (197±12 mL/g). The 80 rpm batches showed a similar trend to the lower-mixing counterparts. However, primarily only one set, 80 rpm at 6.4 KLux-×4, clearly showed the formation of OPGs, and that set resulted in SVI₅<100 mL/g. The settleability of this set was concordant with 6.4 KLux and ×2 dilution and the 12.7 KLux and ×2 dilution batches with SVI₅ at 109 mL/g and 105 mL/g, respectively. As with the 20 rpm and 50 rpm batches, there was little change in SVI₅ for undiluted sets at 80 rpm agitation.

Temporal Change in SVI₅/SVI₃₀ Ratio

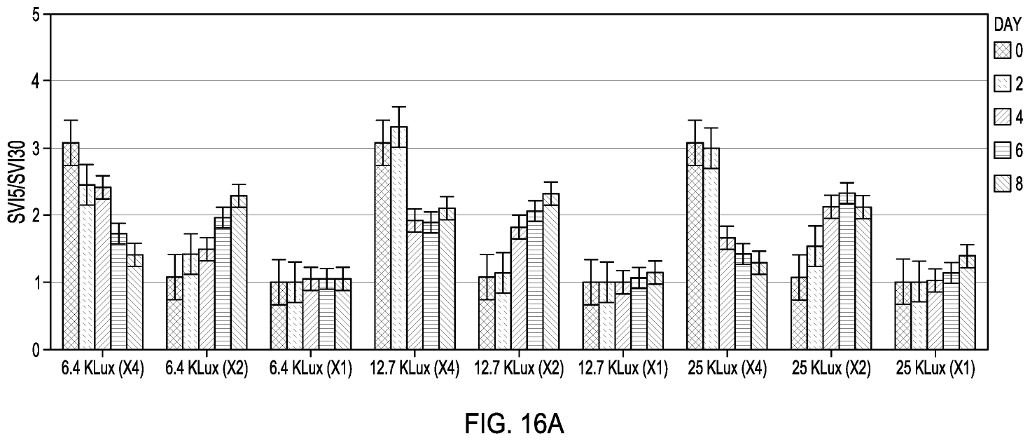

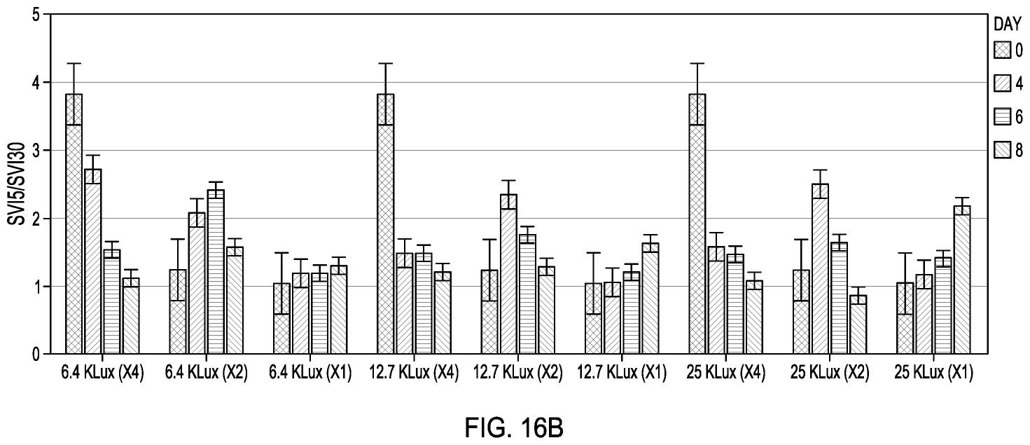

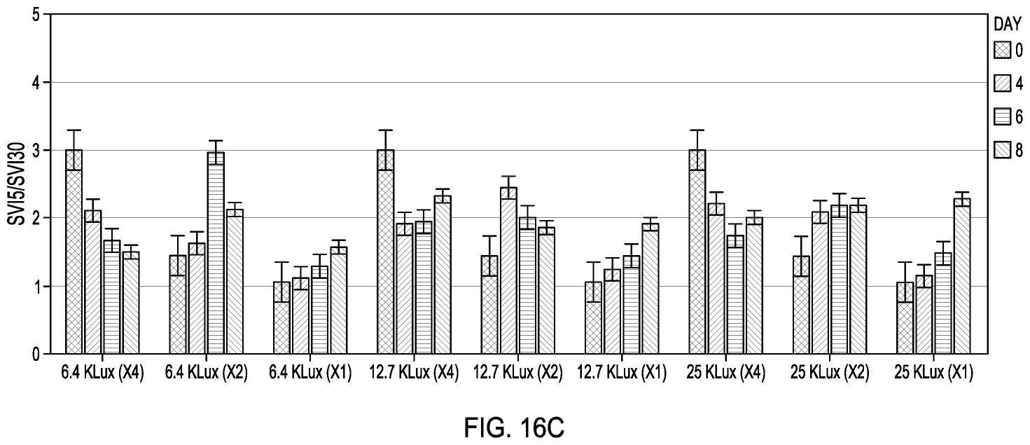

A temporal increase in settling velocities of biomass ensues with transition from floc to granular morphology, which translates to both decrease and convergence of the ratio of SVI₅/SVI₃₀ over the batch period. Therefore, this ratio was examined as a characteristic metric for granulation. FIGS. 16A, 16B, and 16C show the SVI₅/SVI₃₀ ratio for the 20 rpm samples, the 50 rpm samples, and the 80 rpm samples, respectively, measured after 0 days, 2 days, 4, days, 6 days, and 8 days of incubation.

As can be seen in the data of FIGS. 16A-16C, batches with ×4 dilute inoculums in 20 rpm and 50 rpm mixing under all three light conditions, which produced easily observable OPGs, showed clear decreases in the SVI₅/SVI₃₀ ratio over the 8-day experimental period. The decrease from the peak to the day 8 ratio in these six sets was by 60±6%. The counterparts in 80 rpm mixing showed an average decrease of 36±8%, while 80 rpm set with 6.4 KLux and ×4 dilution showed the highest difference at 50%.

The ×2 dilutions in 20 rpm under all light conditions showed increases in SVI₅/SVI₃₀ over the batch period. Although OPGs were formed in these sets (as can be seen in FIG. 8), and the settleability of biomass significantly improved, as seen with 66±4% decrease in SVI₃₀ (FIGS. 15A-15C), both biomass morphology and SVI₅/SVI₃₀ ratio data suggest that granulation in these sets was weaker than the ×4 dilution sets. In the 2× dilution and 50 rpm sets, where photogranules were formed under all light conditions, the SVI₅/SVI₃₀ ratio decreased by day 8 after an initial increase. The average difference between the peak and the day 8 ratios was 48±9%. The 80 rpm and ×2 dilution sets showed weaker convergence in the SVI₅/SVI₃₀ ratio, compared to 50 rpm counterparts, or increased over the batch period.

Undiluted sets under all mixing and light conditions had increasing or unchanging SVI₅/SVI₃₀. These are the batches that also showed no or little change in SVI₅ during the experimental period (as shown in FIGS. 14A-14C). Furthermore, both macroscopic and microscopic examinations did not reveal observable granules. These results suggest that the undiluted batches were the least favorable for photogranulation to occur regardless of the conditions of mixing and light provided.

With the SVI₅/SVI₃₀ ratio as a characteristic metric for granulation, multiple regression models were fit to the change in SVI₅/SVI₃₀ ratio data over the batch period to evaluate the significance and dependence on the experimental parameters of light, mixing, and dilution. The results of the multiple regression fit are summarized in Table 3.

TABLE 3
Multiple regression fit parameters for SVI₅/SVI₃₀ ratio change

| | Std. | | | |
|---|---|---|---|---|
| SVI day | Dev (S) | R² | R² (adj) | R² (pred) |
| Day 6 (M) | 0.85 | 49.33% | 46.35% | 44.06% |
| Day 6 (Mⁱ) | 0.79 | 56.21% | 53.63% | 52.02% |
| Day 8 (M) | 0.81 | 62.17% | 59.94% | 58.81% |
| Day 8 (Mⁱ) | 0.73 | 69.54% | 67.11% | 64.89% |

Model fits without interactions (M) had an R² of 49% on day 6 and 62% on day 8. On the other hand, the model results with parameter interactions (M′) had significant fit (p<0.05) with R² of 56% and 70% for day 6 and day 8, respectively. Interaction of the predictors improved the model fit with lower deviation and better model fit for the data (R²), suggesting significance of their interdependence on the SVI ratios. Moreover, improvements were also seen on the model fit adjusted for additional terms (R² (adj)) and in the model predictive capacity (R² (pred)). This improvement was, on average, 16% (std. dev 0.21) on day 6 and 11% (std. dev 0.01) on day 8 in the R² measures. On day 6 only mixing and dilution interactions showed significant impact on the SVI ratio changes and model-fit coefficients. However, day 8 showed significant interaction by all three parameters—model details are provided in supporting information. The inventors believe that this can be attributed to continued illumination altering the phototrophic composition of biomass and impacting the settleability and granulation.

Phototrophic Enrichment

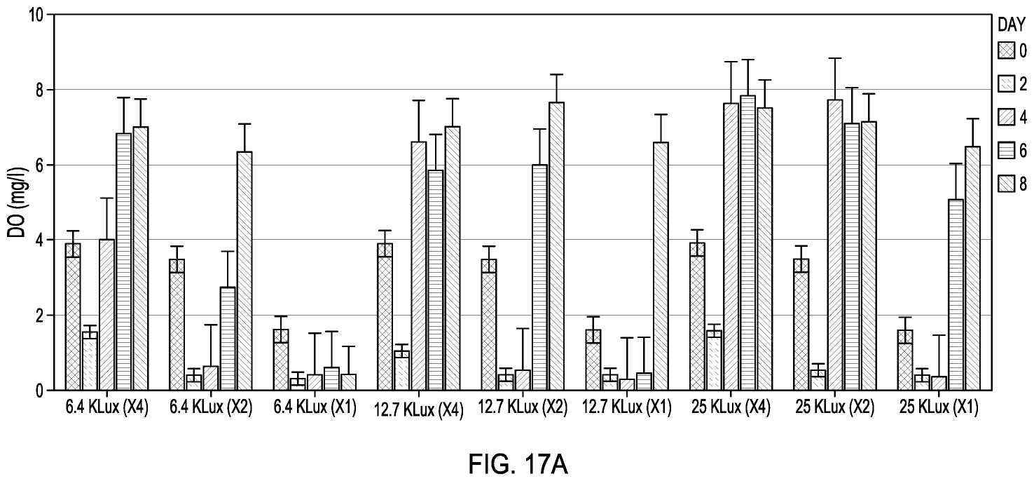

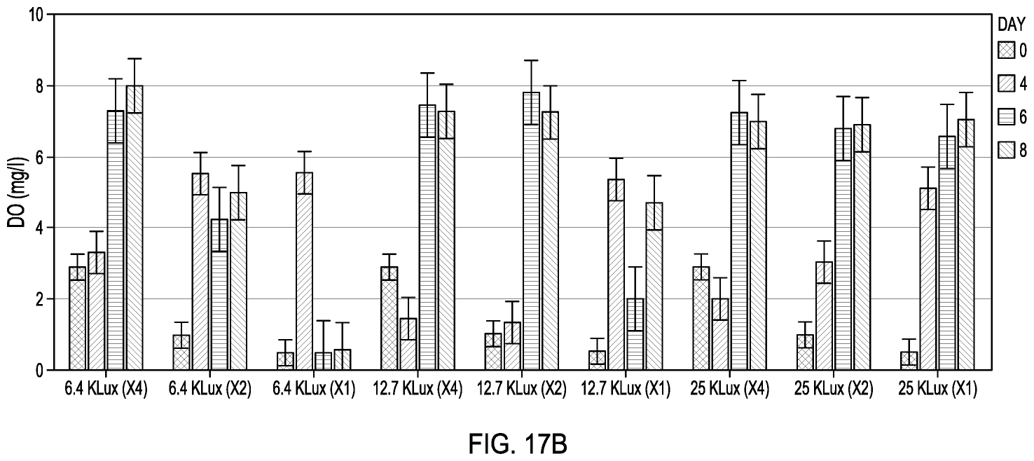

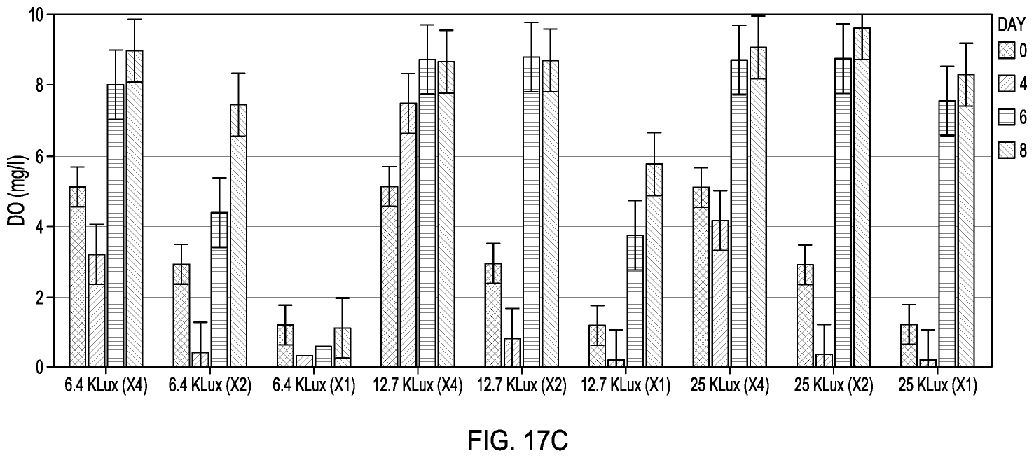

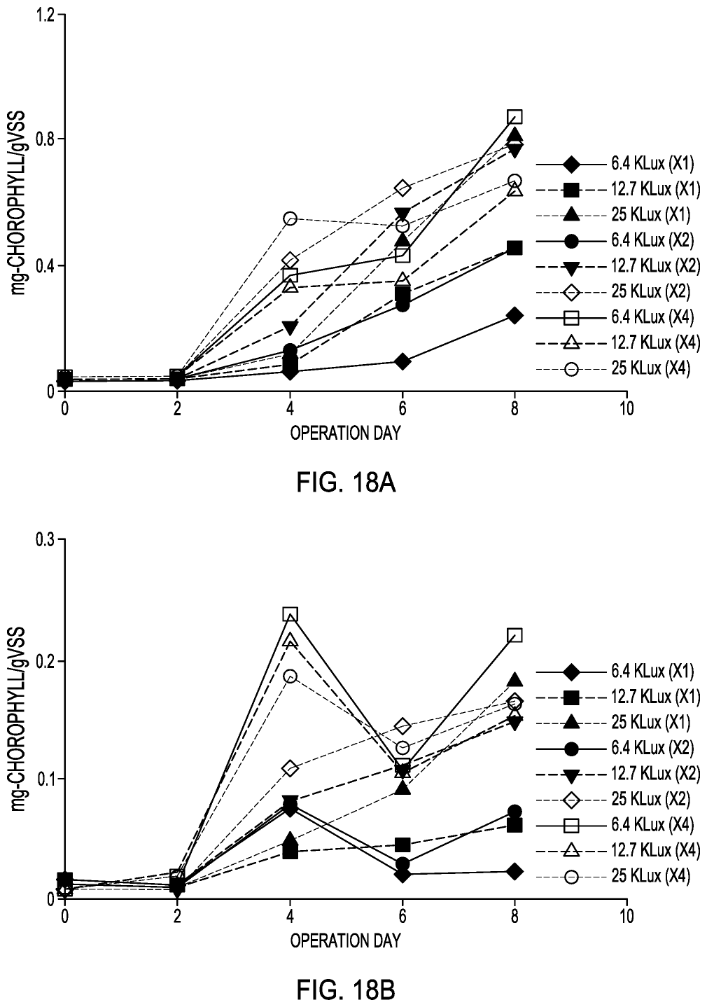

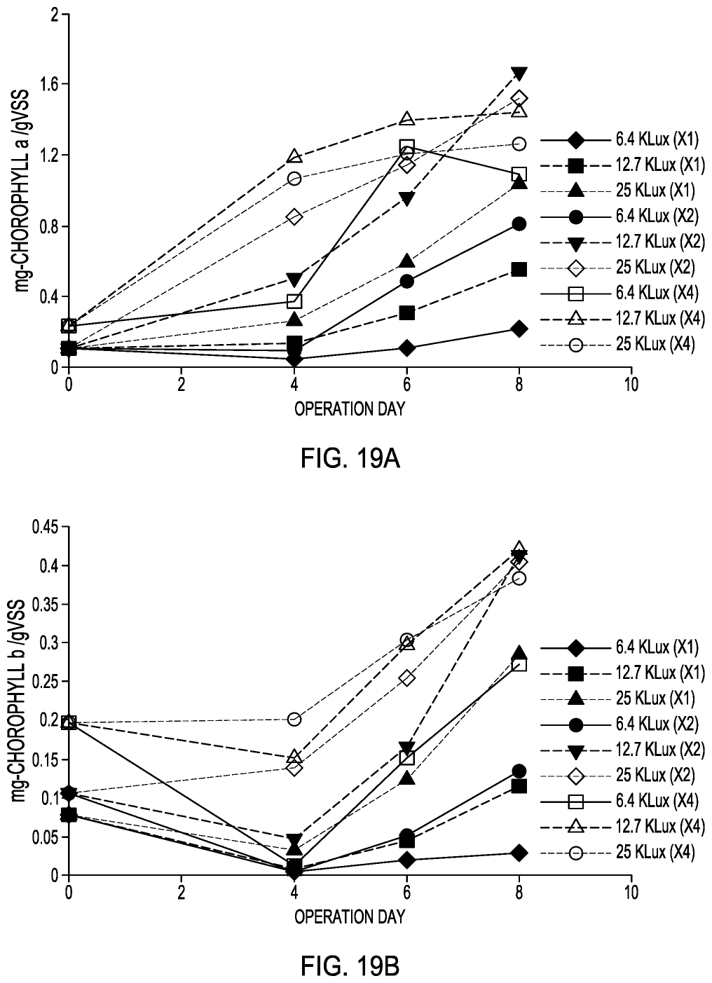

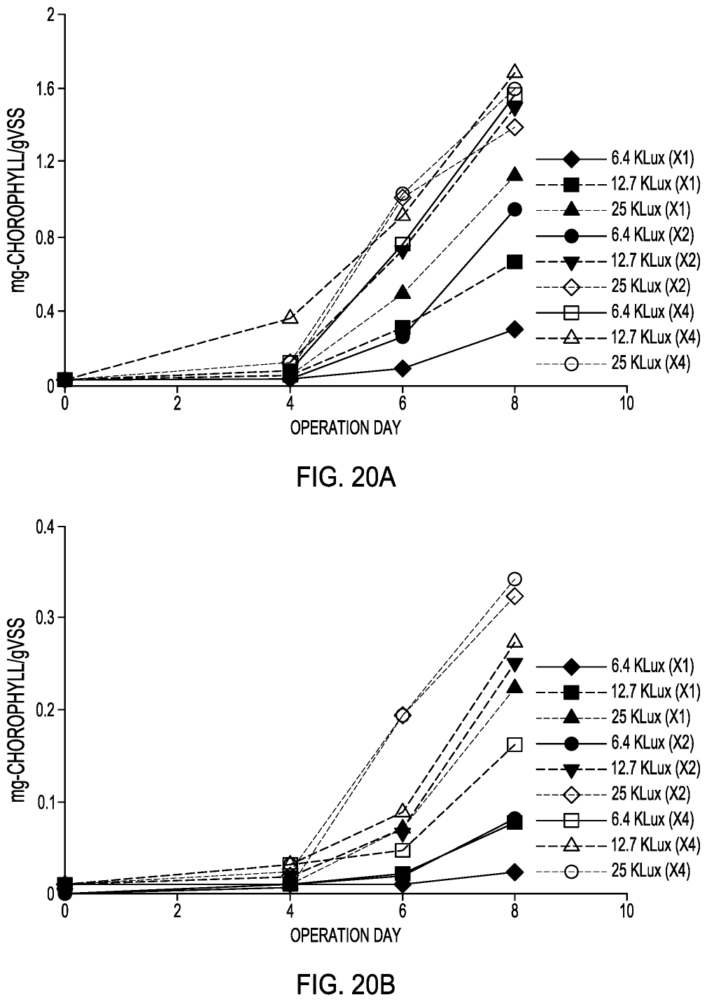

FIGS. 17A, 17B, and 17C show the amount of dissolved oxygen (DO), in mg/L, for the 20 rpm samples, the 50 rpm samples, and the 80 rpm samples, respectively, measured after 0 days, 2 days, 4, days, and 6 days of incubation. FIGS. 18A and 18B show the evolution of the phototrophic pigments chlorophyll a and chlorophyll b, respectively, for the 20 rpm batches; FIGS. 19A and 19B show the evolution of chlorophyll a and chlorophyll b, respectively, for the 50 rpm batches; and FIGS. 20A and 20B show the evolution of the phototrophic pigments chlorophyll a and chlorophyll b, respectively, for the 20 rpm batches.

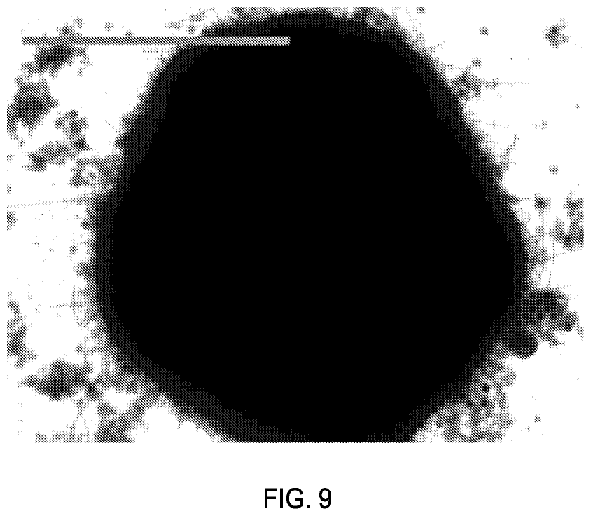

For the 20 rpm sets, each batch was characterized by an initial decay phase with decreasing DO (FIG. 17A). This phase was followed by a phototrophic bloom seen with increasing chlorophyll pigments to day 4 (FIGS. 18A and 18B) and increase in DO indicating photosynthetic oxygenation. Between days 4 and 6, the three ×4 dilution sets, which clearly supported granulation, showed a plateau phase for chlorophyll a but clear declining phase for chlorophyll b. As will be appreciated by those having skill in the art, chlorophyll a is the essential pigment for all phototrophs, while chlorophyll b is an accessory pigment associated with eukaryotic phototrophs. This result, along with microscopic analysis (FIGS. 9 and 10), suggests enrichment of cyanobacteria in these batches. Between days 6 and 8, an increase in both chlorophyll a and b was observed, inferring increased population of microalgae. In the other 20 rpm sets, a consistent increase of chlorophyll a and b ensued between days 4 to 8, in the majority of sets, suggesting prevalence of microalgal enrichment.

For sets under 50 rpm agitation, a general increase in chlorophyll a by day 4 was accompanied by minor changes in chlorophyll b (FIGS. 19A and 19B). These chlorophyll trends allude to cyanobacterial enrichment in the ensemble. The chlorophyll an increased beyond day 4 to the end of the experiment, generally increasing with dilution to day 6. Moreover, chlorophyll a in the ×4 and ×2 sets, those producing OPGs, were clustered higher than non-granulating undiluted sets by day 6—one exception was the 6.4 KLux-2× batch. Chlorophyll b concentrations increased with dilution and light intensity after day 4 in contrast to 20 rpm sets, which primarily increased after day 6. These faster increases, an indicative of faster microalgal growth, can be ascribed to elevated light energy interactions per particle concentration from higher agitation.

Under 80 rpm, both chlorophyll a and b concentrations in most sets had marginal increases up to day 4 and further increases up to day 8 (FIGS. 20A and 20B). Analogous to the 50 rpm ensemble, the increase was proportional to dilution alluding to light penetration expediency within the batch vessels. The pigment concentrations for the ×2 and xl dilutions increased with light intensity but not in ×4 dilution sets, which had potentially higher variability in light interactions. In this 80 rpm set, the trends in chlorophyll a concentrations were strongly correlated to that of chlorophyll b with an average r=0.97, indicating that microalgal growth was dominant in these batches.

### Discussion

This experiment examined the potential for hydrodynamic granulation of OPGs from activated-sludge inoculum. While different combinations of conditions were examined, the batch sets having 20 rpm and 50 rpm mixing, combined with ×4 and ×2 dilution of the activated-sludge inoculum and the three different light conditions tested were found to be amenable for formation of OPGs (FIG. 8). These results present not only an additional way to produce seed OPGs but also opportunities for rapid and bulk development of OPGs compared to the previous hydrostatic cultivation.

The data shows that high agitation rates with 80 rpm mixing (0.07 N/m²; 73 s⁻¹) resulted in a decline of particle sizes and curtailed aggregation for the 1 L reaction vessel, in contrast to the lower agitation rates (FIGS. 11-13; Table 2). However, when combined with low-light intensity 6.4 KLux and ×4 dilution, OPGs were formed even at this high shear. The batch sets with 20 rpm and 50 rpm mixing, on the other hand, resulted in granulation under a broader range of light intensities and dilution. While no OPGs were observed in hydrodynamic batches with undiluted inoculum regardless of any combination with mixing and light conditions provided, OPG granulation has occurred with undiluted activated-sludge inoculum under negligible kinetic energy (i.e., hydrostatic photogranulation). These variable experimental outputs, therefore, indicate a granulation promoting confluence of diverse magnitudes of applied variables (i.e., kinetic, biochemical, and light energies).

Various investigators have reported on the importance of shear as a core selection pressure for granulation. Hydrodynamic forces distribute the mass flux within the reactor and initiate particle-particle interaction with induced shear sculpting the resultant three-dimensional structure. Moreover, the increase in hydrophobicity induced by strong shear is essential for the initial cell-to-cell contact as it reduces the surface free Gibbs energy of the cells, resulting in their separation from the liquid phase. The changes in biomass morphology and the decrease and convergence of SVI₅/SVI₃₀ ratio indicate sustained aggregation of biomass in the lower shear environment compared to the high shear rate 80 rpm sets for this small-volume vessel experiment. Propagation of OPGs has been reported in reactor operations with a calculated shear rate of 38 s⁻¹, comparable to 50 rpm conditions employed in this experiment. This similarity suggests an ideal shear threshold for granulation in a hydrodynamic environment.

The effect of shear on photogranulation was also analyzed from the perspective of particle size evolution. The 80 rpm mixing in this experiment resulted in a calculated Eddy length scale of 119 μm, comparable to or smaller than the inoculum's mean and median particle size of 118 μm and 220 μm, respectively (Table 2). Enhanced particle collisions and dissipation of kinetic energy, therefore, most likely limited aggregation in the rpm ensemble and decreased the bulk consortia size. This shear effect will be particularly enhanced in undiluted sets because increasing solids concentration distorts the viscosity of the bulk fluid transforming its rheology from a Newtonian dominated flow into a particle-particle interaction flow suspension. A higher viscosity in undiluted sets would therefore result in higher shear stress on particles compared to diluted sets, which is supported by PSD trends (FIGS. 11-13) and average mean and median particle sizes among the 80 rpm ensemble (Table 2). This trend also holds true for the lower mixing sets. Nevertheless, the inventors believe that some granulation observed under 80 rpm mixing can be attributed to the microbial resistance to particle interactions and shear stress, especially with dilution. For rpm batches, an Eddy length of 164 μm was determined with a mean inoculum particle size of 132 μm and a median of 220 μm. On the other hand, the 20 rpm set had an Eddy scale of 301 μm compared to the inoculum mean size of 72 μm and median 153 μm. The higher length scales of shear energy dissipation compared to particle sizes and a reduced particle-particle collision could explain the enhanced granulation in the 20-50 rpm ensembles.

In OPGs, the light substrate provides essential photon energy for photosynthesis to occur. Among various phototrophs, enrichment of the subsection III filamentous cyanobacteria possessing motility and their entanglement have been postulated to be responsible for OPG development. In the batch operation adopted, the utility of light substrate is a function of light intensity provided as well as both dilution and agitation. Phototrophic enrichment showed a high sensitivity to increasing light intensity (FIGS. 18-20). Inoculum dilution likewise allows for penetration of light, increasing light-biomass interaction compared to undiluted sets at the same mixing speed. Supporting this, the change in mean sizes had a high positive correlation to the light intensity (r>0.85) across all mixing speeds and dilution (r>0.92). In addition, a higher mixing speed increases the incidence of light exposure at the same light intensity. Consequently, the proportion of phototrophic enrichment generally increased with both the dilution and mixing speed under the same intensity of light.

However, the formation of OPGs in high-shear and high-light substrate conditions (80 rpm-25 KLux) in a small-volume batch was limited (FIG. 8). The high-energy inflow from high agitation and light intensity were found to favor microalgae growth rather than filamentous cyanobacteria, seen with both microscopy (FIG. 10) and the trend of chlorophyll a and b concentrations (FIGS. 18-20). Algae are known for higher shear tolerance compared to both cyanobacteria and dinoflagellates, which may explain the persistence of algae under the 80 rpm agitation. Moreover, microalgae are tolerant and can adapt to high-intensity light, filamentous cyanobacteria are well known for their photophobic characteristic. Hence, the lack of granulation of OPGs in these so-called “high-energy” sets for a small-volume hydrodynamic batch could be due to repressed growth of cyanobacteria, which may arise from photoinhibition and shear-induced limitation.

Despite the different morphologies of biomass, both activated sludge and granular biomass systems employ similar inputs, namely, agitation, a wastewater stream, and a microbial consortium. In conventional activated sludge operations for wastewater treatment, no spontaneous granule formation has been reported to date. However, altered operational conditions have resulted in the formation of granular biomass using activated-sludge inoculum. Moreover, similar inputs exist in other environments, such as in waterways, where niche colonization takes the morphology of mats and biofilms. Thus, despite the prevalence of analogous conditions, the form and interactions of those conditions likely select for enhancement of different phenotypes. It thus seems evident that both operational conditions and ecological and physiological response are culpable for granulation.

It can be surmised that granulation of OPGs, or any granulation, occurs under the influence of macro inputs coupled with biological responses. Results presented with OPG granulation in this experiment indicate the existence of multiple granulation frontiers with different combinations of energy inputs: light, shear, and biochemical energies. This statement also applies to a previous research, which found that granulation of OPGs occurs even in a hydrostatic environment. The various ensembles of energy can be presented as a “zone of granulation interactions.” When these “goldilocks conditions” are achieved, a bio-structural response is expected to ensue favoring spheroidal structures and enhancing selection of aggregating characteristics of filamentous cyanobacteria for OPGs.

Without wishing to be bound by any particular theory, the inventors believe that particle-particle attachment coupled with ecological associations as microbes seek a survival niche occurs at the onset of granulation. The inventors further believe that this initial aggregation is compounded by increasing sizes of the micro flocs due to microbial growth, particle adherence, and filamentous entanglement. Thereafter, it is believed that microbial translocation aided by cell motility and dominance in response to persisting environmental conditions occur in tandem with granular growth. The outcome of experimental EXAMPLE 1 indicates the existence of a wide array of “granulating conditions.”

### Example 2

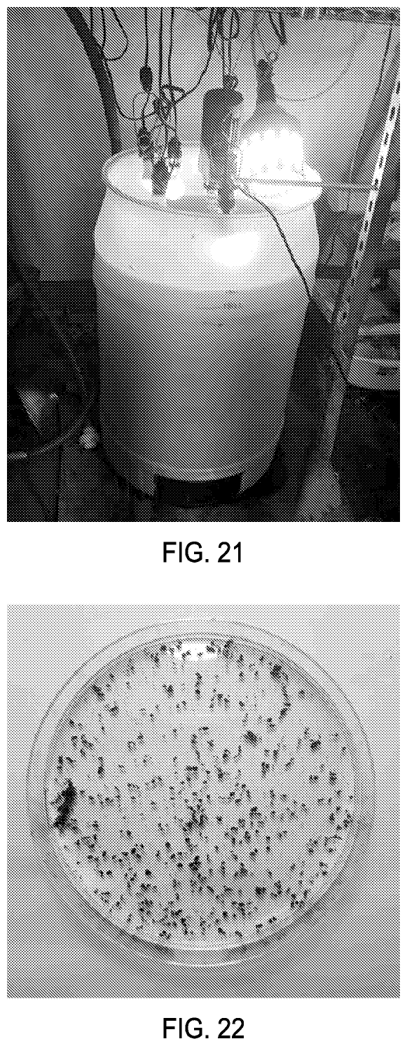

A 190 L hydrodynamic batch experiment was conducted to demonstrate scaling up of the hydrodynamic batch cultivation of seed OPGs from activated sludge as in EXAMPLE 1. Fresh activated sludge collected from a local wastewater treatment plant was diluted with tap water to about 500 mg/L. This diluted activated sludge was placed in a reactor vessel having a working volume of 190 liters (FIG. 21). The diluted activated sludge was illuminated with artificial lighting (shown in FIG. 21) provided a luminous flux onto the reaction mixture of about 450 μmol m⁻² s⁻¹ while agitating the activated sludge with an agitation device (also shown in FIG. 21) at a shear stress of about 0.056 N m′. The large-scale hydrodynamic batch of EXAMPLE 2 produced OPGs. Some of the produced OPGs were recovered on day 14. FIG. 22 shows a photograph of a petri dish containing the OPGs that were recovered on day 14 of the experiment of EXAMPLE 2.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A method comprising the steps of:
placing in a vessel a mixture having a specified suspended solids or sludge concentration and comprising a water-based reaction medium and at least one microalgae including filamentous cyanobacteria, said water-based reaction medium comprising a nutrient material that is consumable by a live bacterium or by a live protozoan present in said water-based reaction medium; and
incubating said mixture for a specified incubation period under at least intermittent illumination with a specified luminous flux during periods of illumination while mixing said mixture under a specified shear stress, wherein said filamentous cyanobacteria forms a supporting matrix that incorporates said live bacterium or said live protozoan into a biologically-active bioaggregate granule, wherein said incubating produces a plurality of said biologically-active bioaggregate granules.

2. A method according to claim 1, further comprising recovering at least a portion of said plurality of biologically-active bioaggregate granules from said incubated mixture.

3. A method according to claim 1, wherein said specified luminous flux as a photosynthetic photon flux density onto the mixture is from about 30 μmol m⁻² s⁻¹ to about 1200 μmol m⁻² s⁻¹.

4. A method according to claim 1, wherein said specified shear stress is from about 0.005 Newtons per square meter to about 0.075 Newtons per square meter.

5. (canceled)

6. A method according to claim 1, wherein said specified suspended solids or sludge concentration is from about 30 milligrams of said suspended solids or said sludge per liter of said mixture to about 1,500 milligrams of said suspended solids or said sludge per liter of said mixture.

7. (canceled)

8. (canceled)

9. A method according to claim 1, further comprising:
receiving an activated sludge; and
diluting said sludge with water to a concentration of from about 30 milligrams of said activated sludge per liter of said water to about 1,500 milligrams of said activated sludge per liter of said water to provide said water-based reaction medium.

10. A method according to claim 1, wherein said specified incubation period is no more than about 15 days.

11. (canceled)

12. A method according to claim 1, wherein said specified incubation period is from about 3 days to about 12 days.

13. (canceled)

14. A method according to claim 1, wherein said nutrient material is consumable by said at least one microalgae including filamentous cyanobacteria.

15-19. (canceled)

20. A plurality of said biologically-active bioaggregate granules made according to the method of claim 1.

21. A method of wastewater remediation, comprising the steps of:
adding a first portion of said plurality of said biologically-active bioaggregate granules made according to claim 1 into a wastewater treatment system;
receiving wastewater having a first amount of biologically-active waste per unit volume into said wastewater treatment system; and
operating said wastewater treatment system under operating conditions that allow said first portion of said plurality of said biologically-active bioaggregate granules to consume a portion of said biologically-active waste to provide a processed wastewater having a second amount of biologically-active waste per unit volume, said second amount being lower than said first amount.

22. A method according to claim 21, further comprising recovering from said wastewater treatment system at least a portion of said processed wastewater.

23. (canceled)

24. A method of generating biomass, the method comprising the steps of:
adding a first portion of said plurality of biologically-active bioaggregate granules made according to the method of claim 1 into a wastewater treatment system;
operating said wastewater treatment system under operating conditions that allow said first portion of said biologically-active bioaggregate particles generate an additional quantity of said biologically-active bioaggregate granules that is more than said first portion; and
recovering from said wastewater treatment system at least some of said additional quantity of said biologically active bioaggregate granules, leaving in said wastewater treatment system a sufficient amount of said biologically-active bioaggregate granules to continue operation of said wastewater treatment system.

25. A system for cultivating oxygenic photogranules for wastewater treatment, the system comprising:
a reaction vessel for receiving a mixture comprising a water-based reaction medium having a specified suspended solids or sludge concentration and comprising live bacterium or live protozoan, a nutrient material that is consumable by said live bacterium or by said live protozoan, and at least one microalgae including filamentous cyanobacteria;
an illumination source configured to illuminate said mixture at least intermittently with a specified luminous flux during periods of illumination for a specified incubation period; and
an agitator configured to mix said mixture under a specified shear stress during said specified incubation period;
wherein said illumination of said mixture at said specified luminous flux while mixing said mixture under said specified shear stress during said incubation period incubates said mixture such that said filamentous cyanobacteria forms a supporting matrix that incorporate said live bacterium or said live protozoan to provide a plurality of biologically-active bioaggregate granules.

26. A system according to claim 25, further comprising a separator to recover at least a portion of said plurality of biologically-active bioaggregate granules from said incubated mixture.

27. A system according to claim 25, wherein said specified luminous flux is from about 30 μmol m⁻² s⁻¹ to about 1200 μmol m⁻² s⁻¹.

28. A system according to claim 25, wherein said specified shear stress is from about 0.005 Newtons per square meter to about 0.075 Newtons per square meter.

29. A system according to claim 25, wherein said specified suspended solids or sludge concentration is from about 30 milligrams of said suspended solids or said sludge per liter of said mixture to about 1,500 milligrams of said suspended solids or said sludge per liter of said mixture.

30-32. (canceled)

33. A system according to claim 25, wherein said specified incubation period is no more than about 15 days.

34-36. (canceled)

37. A system according to claim 25, wherein said nutrient material is consumable by said at least one microalgae including filamentous cyanobacteria.

38-42. (canceled)
